(19) United States
(12) Patent Application Publication
(10) Pub. No.: US 2026/0279518 A1
(43) Pub. Date: Sep. 17, 2026

(54) SYSTEMS AND METHODS FOR PRODUCING BATCH RECORDS FOR PHARMACEUTICAL TRIALS

(71) Applicant: R.P. Scherer Technologies, LLC, Carson City, NV (US)

(72) Inventors: Carsten STAHLHOFEN, Eppstein (DE); Samantha Jane JONES, Flintshire (GB); Philip ZEMLER, Olathe, KS (US); Anishsinh RATHOD, Riverview, FL (US)

(73) Assignee: R.P. Scherer Technologies, LLC, Carson City, NV (US)

(21) Appl. No.: 19/480,125

(22) PCT Filed: Apr. 30, 2024
(86) PCT No.: PCT/US2024/026973

§ 371 Date: Oct. 30, 2025

Related U.S. Application Data
(60) Provisional application No. 63/463,183, filed on May 1, 2023.

(51) Int. Cl. G16H 10/20; G06Q 10/0631

(52) U.S. Cl. CPC G16H 10/20; G06Q 10/063114

(57) ABSTRACT

Embodiments of the present disclosure include systems and methods for producing an electronic batch record.

### CROSS-REFERENCE TO RELATED APPLICATIONS

This application claims the benefit of U.S. Provisional Application No. 63/463,183 filed May 1, 2023, the entire contents of which are incorporated herein by reference.

### FIELD

The present disclosure relates generally to systems and methods for producing batch records for clinical trial studies, and more specifically, to systems and methods for producing electronic batch records for output materials for clinical trial studies.

### BACKGROUND

Companies engaging in clinical trial studies, particularly, pharmaceutical clinical trials are required to adhere to government regulated clinical packaging and labeling standards. For example, various national bodies like the Food and Drug Administration (FDA), Medicines and Healthcare products Regulatory Agency (MHRA) and European Medicines Agency (EMA) provide a regulatory framework for clinical packaging. As such, each clinical packaging job, whether a primary (e.g., putting bulk drug into bottle, blister, vial or similar) or a secondary packaging job (e.g., clinically labeling a primary bottle and putting it into a box and labeling the box to create a patient kit), must be controllable and repeatable in action and follow appropriate regulatory guidelines. Moreover, the packaging should be performed in a way that does not reveal which patient kits are associated with which treatment types (e.g., does not unblind the clinical trial by allowing a participant or another individual involved with the clinical trial to discover whether a kit is an active treatment or a placebo).

Traditionally, a batch record for a clinical trial is prepared in the form of a paper batch record. Upon receiving pre-approval from one or more individuals overseeing the clinical trial, the paper batch record may then be disseminated to one or more packaging sites and filled in manually by operators during primary or secondary packaging runs. Additional paperwork may be generated during the packaging process may be added to the batch record to produce an executed batch record. This paper-based process, however, relies heavily on manual input, which can lead to human error, and does not produce standardized batch records across sites. Furthermore, since all fields in a paper batch record are visible to anyone viewing the batch record during packaging, this may reveal which patient kits are associated with which treatment types, thereby unblinding the clinical trial. Additionally, adjustments and approval on the packaging floor may be made during the packaging process. These adjustments and approval at various stages of the packaging process are documented via a paper process, where such documentation may be referred to during later packaging runs or during audits. However, paper documentation typically creates a large volume of paper documents that re difficult to organize and access.

Accordingly, there is a need to efficiently produce electronic batch records corresponding to packaged kits for clinical trials which can provide a harmonized and centralized record across various packaging sites while preserving the blindness of the clinical trials and without disrupting the flow of the packaging process.

### SUMMARY

Embodiments of the present disclosure can provide computational systems and methods for efficiently and accurately producing electronic batch records. For example, embodiments of the present disclosure provide systems and methods that enable users (e.g., operators, team leads, quality assurance personnel) to efficiently and accurately package output materials for packaging runs for one or more clinical trial studies. Embodiments of the present disclosure further provide a centralized system for generating and preserving data throughout the packaging process—from room assignment and ensuring that the packaging is occurring in the proper room with the proper equipment to packaging instructions and documenting various steps of the packaging process to reconciliation and ensuring that the packaged output materials meet predetermined reconciliation limits. Embodiments of the present disclosure further provide a streamlined system that accurately tracks which users to update and approve data for entry into the executed batch record during the packaging process. For instance, each comment and/or approval entered by a user is attributed to the appropriate user based on user credentials as opposed to associating comments and/or approval based on which user is logged into the system. This eliminates the need for multiple users to be logged on to the system and/or to log in and out of user accounts to grant approval during the packaging process. In this manner, embodiments of the present disclosure further provide a system that enhances accountability throughout the packaging process as each step of the packaging process is associated with a corresponding user.

Embodiments of the present disclosure provide computer implemented methods for generating an executed batch record for a clinical trial study. In one or more embodiments, such methods can comprise: receiving an indication that a batch record is assigned to a packaging room located at a packaging site, the packaging room configured to execute the batch record; receiving an indication at the packaging site that a first user completed a package briefing associated with a packaging job to execute the batch record; in accordance with a determination that the first user completed the package briefing, displaying packaging instructions for producing a quantity of packaged materials for the packaging job; determining whether the quantity of packaged materials is within a reconciliation target and updating the batch record to include a determination of whether the quantity of packaged materials is within the reconciliation limit; receiving a first approval of the batch record by the first user, the first approval comprising receiving credentials from the first user while logged into an account associated with the first user; receiving a second approval of the batch record by a second user, the second approval comprising receiving credentials from the second user while remaining logged-in to the account associated with the first user; and generating the executed batch record for the clinical trial study based on the approved batch record.

In some embodiments, the packaging instructions comprise a plurality of steps and the method further comprises: for a step of the plurality of steps, receiving, via an image capture device located in the packaging room, an image comprising a visual indication that the step was completed; and updating the batch record to save the image and associate the image with the step of the plurality of steps. In some embodiments, the method further comprises: for the step of the plurality of steps, receiving an instruction step approval associated with the step by the first user, the instruction step approval comprising receiving the credentials from the first user.

In some embodiments, the method further comprises: obtaining, via an external resource, input material data comprising one or more fields, the materials data associated with an input material staged in the packaging room; in accordance with a determination that one or more fields of the input material data matches one or more corresponding fields of the executable batch record, approving the staged input material; in accordance with a determination that a field of the input material data does not match a corresponding field of the executable batch record, displaying a user affordance for the first user to accept or reject the input material; and in accordance with a determination that the input material is approved or accepted, adding the input material to the batch record in execution. In some embodiments, the one or more fields of the input material data comprises one or more of a material identification number, a supplier lot number, an external resource lot number, and a customer lot number. In some embodiments, the method further comprises: in accordance with receiving an indication that the first user has accepted the input material, displaying an input material approval interface; receiving credentials of the first user via the input material approval interface; and accepting or rejecting the input material.

In some embodiments, the method further comprises: receiving via comment user interface a comment associated with the batch record in execution. In some embodiments, the method further comprises: receiving a comment approval of the comment by the second user, the comment approval comprising receiving the credentials from the second user while remaining logged-in to the account associated with the first user. In some embodiments, the method further comprises: receiving an indication that the comment is associated with blinding data. In some embodiments, generating the executed batch record comprises generating a blinded executed batch record such that the blinding data is redacted.

In some embodiments, the method further comprises configuring the packaging room to execute the batch record, wherein configuring the packaging room comprises: assigning a packaging room at the packaging site to the packaging job; receiving an indication from a user located at the packaging room that the packaging room satisfies one or more items of a room clearance checklist, the room clearance checklist associated the executable batch record and the packaging room; receiving an indication from the user that the packaging room satisfies one or more items of a room setup checklist, the room setup checklist associated the executable batch record and the packaging room; and receiving an indication from the user that a first equipment located in the packaging room satisfies one or more items of an equipment setup checklist, wherein the equipment setup checklist is associated with the first equipment and the executable batch record.

In some embodiments, the method further comprises: receiving a user input to put the packaging job on hold; in accordance with receiving the user input to put the packaging job on hold, displaying an job hold approval interface; receiving credentials of the first user via the job hold approval interface; receiving credentials of the second user via the job hold approval interface while remaining logged-in to the account associated with the first user; and updating a packaging job status to on hold.

In some embodiments, the method further comprises: displaying a color-coded progress bar indicative of completion of one or more tasks associated with the packaging job. In some embodiments, the method further comprises: automatically updating the progress bar when the one or more tasks associated with the packaging job are approved.

In some embodiments, a method for generating an executed batch record for a clinical trial study is provided, the method comprising, at a first computer system comprising one or more processors and one or more memories: obtaining an executable batch record for a packaging job, the packaging job configured to performed at a packaging site; assigning a packaging room at the packaging site to the packaging job; receiving an indication that the packaging room satisfies one or more items of a room clearance checklist, the room clearance checklist associated with the executable batch record and with the packaging room; receiving an indication that the packaging room satisfies one or more items of a room setup checklist, the room setup checklist associated with the executable batch record and with the packaging room; receiving an indication that a first equipment located in the packaging room satisfies one or more items of an equipment setup checklist, wherein the equipment setup checklist is associated with the first equipment and with the executable batch record; receiving an indication that a first user completed a package briefing associated with the packaging job; receiving an indication verifying that one or more materials in the packaging room match one or more materials in a bill of materials; receiving an indication to start the packaging job; in accordance with a determination that the first user completed the package briefing and while logged-in to an account associated with the first user, displaying packaging instructions associated with the packaging run, the packaging instructions comprising a plurality of steps; for a step of the plurality of steps, receiving, via an image capture device located in the packaging room, an image comprising a visual indication that the step was completed; receiving an input quantity indicative of a quantity of packaged materials; verifying that the quantity of packaged materials are within a predetermined reconciliation target; receiving an indication that the packaging job is complete; receiving a first approval of the batch record by the first user, the first approval comprising receiving credentials from the first user while logged into the account associated with the first user; receiving a second approval of the batch record by a second user, the second approval comprising receiving credentials from the second user while remaining logged-in to the account associated with the first user; and generating the executed batch record for the clinical trial study based on the approved batch record.

### BRIEF DESCRIPTION OF THE FIGURES

FIGS. 1A and 1B illustrate an exemplary process for producing electronic batch records, in accordance with some embodiments of this disclosure.

FIG. 1C illustrates an exemplary batch record in execution progress bar associated with an exemplary process for producing electronic batch records, in accordance with some embodiments of this disclosure.

FIGS. 2A-2E illustrate exemplary user interfaces associated with obtaining an executable batch record for a packaging job, in accordance with some embodiments of this disclosure.

FIGS. 3A-3M illustrate exemplary user interfaces associated with assigning a packaging room to a packaging job, in accordance with some embodiments of this disclosure.

FIGS. 4A and 4B illustrate exemplary user interfaces associated with completing a room clearance checklist, in accordance with some embodiments of this disclosure.

FIGS. 5A-5D illustrate exemplary user interfaces associated with completing a room setup checklist, in accordance with some embodiments of this disclosure.

FIGS. 6A-6J illustrate exemplary user interfaces associated with completing equipment setup for a packaging job, in accordance with some embodiments of this disclosure.

FIGS. 7A-7G illustrate exemplary user interfaces associated with briefing users on a packaging job, in accordance with some embodiments of this disclosure.

FIG. 8 illustrates an exemplary user interface associated with verifying that materials in a packaging room match materials in a bill of materials, in accordance with some embodiments of this disclosure.

FIGS. 9A-9H illustrate exemplary user interfaces associated with starting a packaging job and displaying packaging instructions, in accordance with some embodiments of this disclosure.

FIGS. 10A and 10B illustrate exemplary user interfaces for verifying that materials are within reconciliation limits, in accordance with some embodiments of this disclosure.

FIGS. 11A and 11B illustrate exemplary user interfaces for generating an electronic executed batch record for review, in accordance with some embodiments of this disclosure.

FIG. 12 illustrates excerpts from an exemplary electronic executed batch record, in accordance with some embodiments of this disclosure.

FIGS. 13A-13I illustrate exemplary user interfaces for completing an in-progress checklist, in accordance with some embodiments of this disclosure.

FIGS. 14A and 14B illustrate exemplary user interfaces for adding sample data to the batch record in execution, in accordance with some embodiments of this disclosure.

FIG. 15 illustrates an exemplary user interface for adding comments to the batch record in execution, in accordance with some embodiments of this disclosure.

FIGS. 16A-16D illustrate exemplary user interfaces for editing the batch record in execution, in accordance with some embodiments of this disclosure.

FIG. 17 illustrates an exemplary electronic device, in accordance with some embodiments of this disclosure.

### DETAILED DESCRIPTION

Embodiments of the present disclosure can provide computational systems and methods for efficiently producing electronic batch records. For example, embodiments of the present disclosure provide systems and methods that enable users (e.g., operators, team leads, quality assurance personnel) to efficiently and accurately package output materials for packaging runs for one or more clinical trial studies. Embodiments of the present disclosure further provide a centralized system for generating and preserving data throughout the packaging process—from room assignment and ensuring that the packaging is occurring in the proper room with the proper equipment to packaging instructions and documenting various steps of the packaging process to reconciliation and ensuring that the packaged output materials meet predetermined reconciliation limits. Embodiments of the present disclosure further provide a streamlined system that accurately tracks which users to update and approve data for entry into the executed batch record during the packaging process. For instance, each comment and/or approval entered by a user is attributed to the appropriate user based on user credentials as opposed to associating comments and/or approval based on which user is logged into the system. This eliminates the need for multiple users to be logged on to the system and/or to log in and out of user accounts to grant approval during the packaging process. In this manner, embodiments of the present disclosure further provide a system that enhances accountability throughout the packaging process as each step of the packaging process is associated with a corresponding user.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Although the following description uses terms “first,” “second,” etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

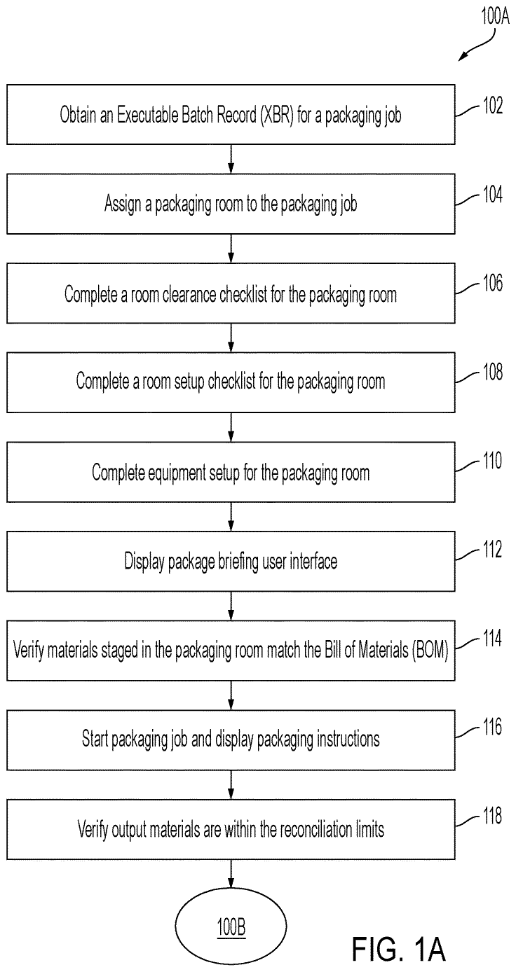

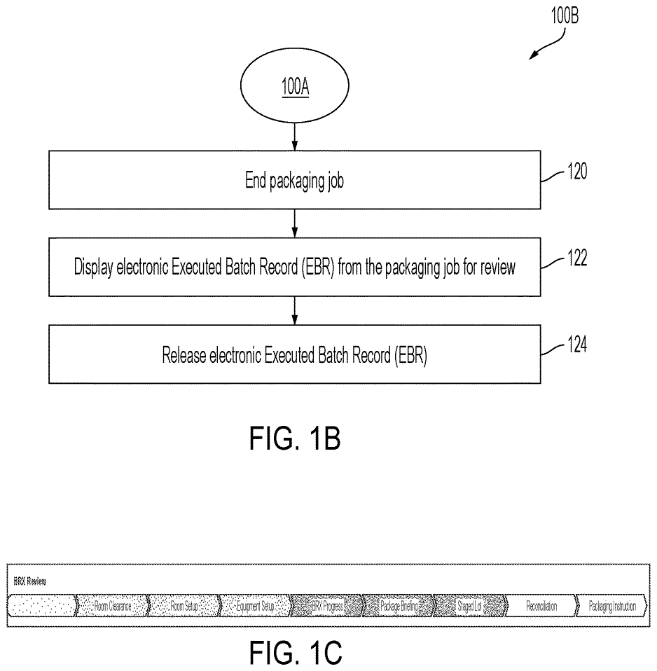

Embodiments of the present disclosure provide a system and method to facilitate the fulfillment of a packaging job corresponding to a batch record in execution. FIGS. 1A and 1B illustrate exemplary processes 100A and 100B for producing electronic batch records. Processes 100A and 100B can be performed, for example, using one or more electronic devices implementing a software platform. In some examples, processes 100A and 100B are performed using a client-server system, and the blocks of processes 100A and 100B are divided up in any manner between the server and a client device. In other examples, the blocks of processes 100A and 100B are divided up between the server and multiple client devices. In other examples, processes 100A and 100B are performed using only a client device or only multiple client devices. In processes 100A and 100B, some blocks are, optionally, combined, the order of some blocks is, optionally, changed, and some blocks are, optionally, omitted. In some examples, additional steps may be performed in combination with processes 100A and 100B. Accordingly, the operations as illustrated (and described in greater detail below) are exemplary by nature and, as such, should not be viewed as limiting.

At block 102 of FIG. 1A, the system can obtain an executable batch record which contains various information fields associated with a supply order. The batch record obtained may be executed as a part of a packaging job. At block 104 of FIG. 1A, the system can present a Room Assignment tab, including a user interface for a user to assign a room to the packaging job. At block 106 of FIG. 1A, the system can present a Room Clearance tab, including a user interface for a user to complete a room clearance checklist. At block 108 of FIG. 1A, the system can present a Room Setup tab, including a user interface for a user to complete a room setup checklist. At block 110 of FIG. 1A, the system can present an Equipment Setup tab, including a user interface for a user to complete an equipment setup process. At block 112 of FIG. 1A, the system can present a Package Briefing tab, including a user interface that includes information for a first user (e.g., operator) to review and/or be briefed by a second user (e.g., Team Lead). In one or more embodiments, the Package Briefing tab may be available for completion by a user upon confirmation that block 102 through 110 have been completed. At block 114 of FIG. 1A, the system can present a Staged Lot Verification tab, including a user interface for a user to verify whether materials staged in the packaging room match the bill of materials (BOM) associated with the batch record in execution (BRX). At block 116 of FIG. 1A, the system can start the packaging job and display packaging instructions. One or more users may fulfill the packaging job in accordance with the packaging instructions. For instance, the one or more users may produce one or more output materials in accordance with the packaging instructions included in the batch record in execution. In some embodiments, a user may capture one or more images of various steps of the packaging process and upload the images to the system. At block 118 of FIG. 1A, the system can verify that the output materials produced are within the reconciliation limits. At block 120 of FIG. 1B, the system can end the packaging job. At block 122 of FIG. 1B, the system can produce an electronic executed batch record (EBR) for review. At block 124 of FIG. 1B, the system can release the executed batch record (EBR) upon completion of the review. Each of these steps will be described in greater detail below.

Embodiments of the present disclosure provide a system and method to facilitate the fulfillment of a packaging job corresponding to a batch record in execution. Embodiments of the present disclosure may display a user interface comprising one or more tabs or pages to facilitate the fulfillment of a packaging job. In accordance with embodiments of the present disclosure the user interface displayed to the user may include a progress bar comprising a summary of one or more steps of the processing job and indicating a status of each of the one or more steps. An exemplary progress bar is illustrated in FIG. 1C. As shown in the figure, the progress bar comprises one or more segments, each segment corresponding to one or more steps of the packaging job, including: Room Clearance, Room Setup, Equipment Setup, BRX (batch record in execution) progress, Package Briefing, Staged Lot, Reconciliation, and Packaging Instruction. More or less steps may be included in the progress bar without departing from the scope of the disclosure.

In one or more examples, the one or more segments may be color coded to indicate a status of the corresponding step. For instance, a segment shaded in green may indicate that a step is complete, a segment shaded in blue may indicate that the step is in progress, and a white segment may indicate that the step has not been started. The system may determine a step is complete if the corresponding step has received approval by one or more users according to the system requirements. The system may determine a step is in progress if the corresponding step has received user input regarding the step (e.g., the user has entered data or updated one or more fields of the user interface associated with the step), but the step has not received the requisite approval. The system may determine a step has not begun if the system has not received user input regarding the step (e.g., the user has not entered data or updated one or more fields of the user interface associated with the step).

In one or more examples, multiple segments of the progress bar may be “in progress.” For instance, as shown in the figure, the BRX progress segment, package briefing segment, and staged lot segments are in progress (e.g., shaded in blue), the room clearance segment, room setup segment, and equipment setup segment are complete (e.g., shaded in green), and the reconciliation segment and the packaging instructions segment have not started (e.g., colored white).

Referring to block 102 of FIG. 1A, the system can obtain an executable batch record (XBR), which contains various information fields associated with a supply order. For instance, the system can receive an input from a user selecting an executable batch record to be executed for a packaging job. Methods and systems for producing an executable batch record are described in greater detail in Application No. PCT/US2022/082479, filed on Dec. 28, 2022, which is incorporated herein in its entirety.

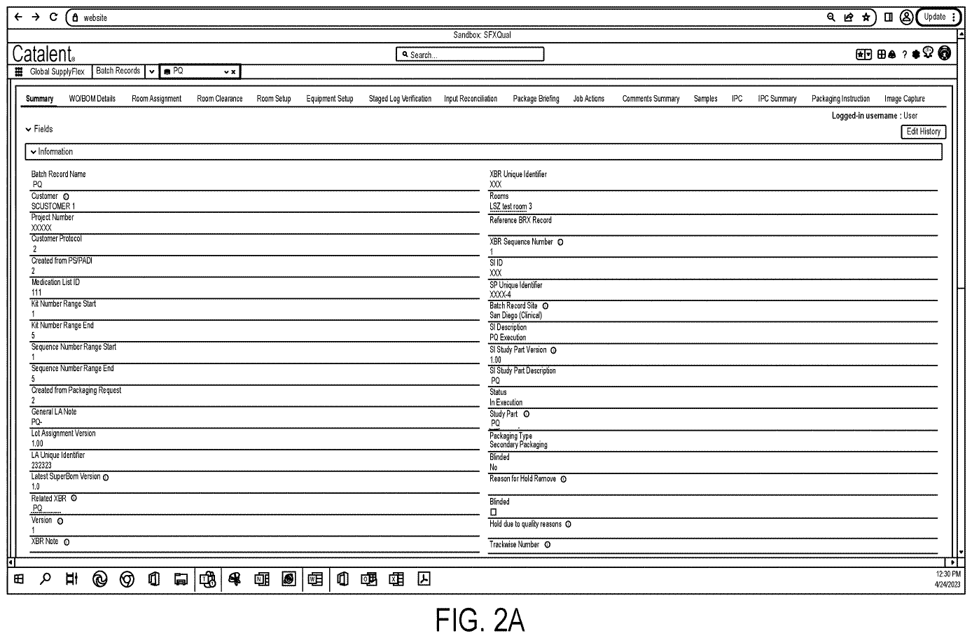

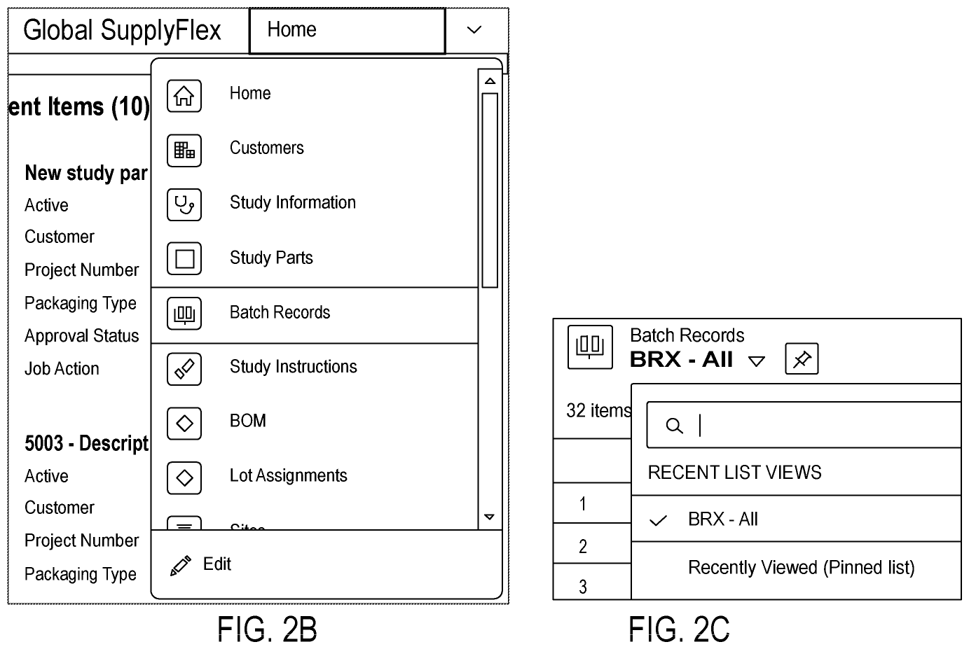

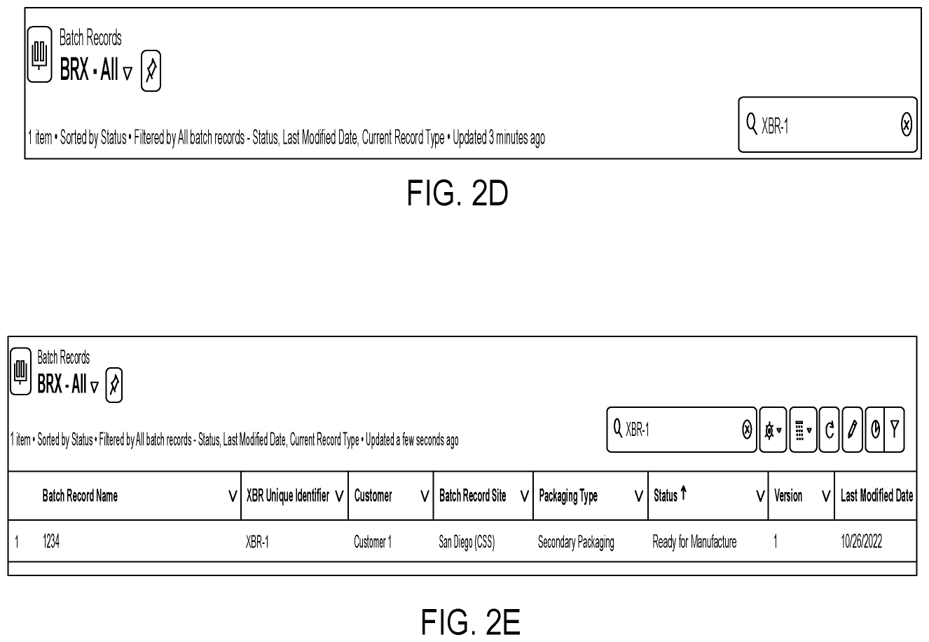

FIG. 2A illustrates an exemplary user interface that shows the executable batch record obtained at block 102. Notably, the executable batch record can contain information associated with preparing and packaging output materials such as kits for the clinical trials. That is, the executable batch record can be executed using a system in accordance with embodiments of this disclosure to package the output materials. For instance, the executable batch record includes information such as packaging instructions, equipment groups, room assignments, whether the study is blinded, and the like. As shown in FIG. 2A, the Summary tab of the executable batch record may display various information fields, including, but not limited to, batch record name, customer name, project number, packaging site, medication type, kit details, document versions, related executable batch records, unique identifiers, packaging room name, attached notes, status, study part, packaging type, blinded status, and hold status. The type of information shown in FIG. 2A is exemplary and more or less information may be associated with an executable batch record without departing from the scope of this disclosure.

The executable batch records available for display to a user is determined based on the user's permissions for a given site. As used herein, a user may correspond to an individual assisting with packaging the output materials. The user may be associated with one or more roles, including, but not limited to: an operator, a team lead, quality assurance personnel, and the like. The user may be associated with other roles without departing from the scope of this disclosure. A user's access permissions within the system may be determined, in part, based on their role. The access permissions may refer to one or more actions a user is permitted to take within the system, e.g., but not limited to, inputting information and providing approval. In one or more examples, a user may be associated with a particular packaging site. For example, if a user associated with a packaging site in Philadelphia may have access to executable batch records for the Philadelphia site but not batch records for another site, e.g., Kansas City. Accordingly, a list of available executable batch records for viewing by the user will include executable batch records assigned to be packaged at the Philadelphia site.

To view a desired executable batch record, the system may receive from a user a selection of the “Home” dropdown menu on the user interface, and further receive a selection of the “Batch Records” option, as shown in FIG. 2B. This opens the Batch Records tab. On this tab, the system may receive a user selection of the dropdown menu beneath “Batch Records,” and a user selection of the “BRX-All” option, as shown in FIG. 2C. Making this selection will display the approved executable batch records and a corresponding status, (e.g., whether a batch record is available for execution and/or in execution). In some embodiments, the system may permit a user to perform a search for a specific batch record. For instance, the system may present a “Search” box, as shown in FIG. 2D, where a user may enter the identifier associated with the batch record. The system can display the search results via the user interface, and receive a selection of a batch record by the user (e.g., the user can select and/or open the batch record for execution by clicking the name of the desired batch record), as shown in FIG. 2E.

The batch record selected for execution may transition from being an “executable batch record” (XBR) to a “batch record in execution” (BRX). A batch record in execution refers to a batch record that is in the process of being executed in a packaging run (e.g., packaging job). Referring back to block 104 of FIG. 1A, the system can present a Room Assignment tab, including a user interface for a user to assign a room to the packaging job. A packaging site may comprise multiple rooms for packaging output materials for fulfilling the executable batch record. Packaging sites may fulfill multiple packaging jobs that span multiple days, and each packaging room is dedicated to a single corresponding packaging job at a time. Assigning a room to a particular packaging job avoids room conflicts and ensures new room assignments do not disrupt ongoing packaging jobs. Moreover, in addition to ensuring that a room is available for a packaging job (e.g., not assigned to another packaging job) or assigning a room to a packaging job in block 4 may include ensuring the room includes the appropriate equipment for completing a particular executable batch record. Thus, the room assignment ensures that a room assigned to the batch record in execution is available for packaging and includes the necessary equipment for fulfilling the executable batch record.

At the packaging site, each of the packaging rooms may be labeled with a room number and a barcode. The system may access data regarding the room via master data of the system. The master data can include information regarding the room setup, e.g., room processing conditions—including, but not limited to, temperature, humidity, and light conditions. The master data of the system can further include information regarding the available equipment in the room. As used herein, the term “master data” can refer to data that is used to create and maintain information associated with one or more customers (e.g., companies that running clinical trials). The master data can include, but is not limited to customer information, packaging site information (e.g., room setups), instruction sets, packaging instructions, equipment information, reconciliation limits, and studies.

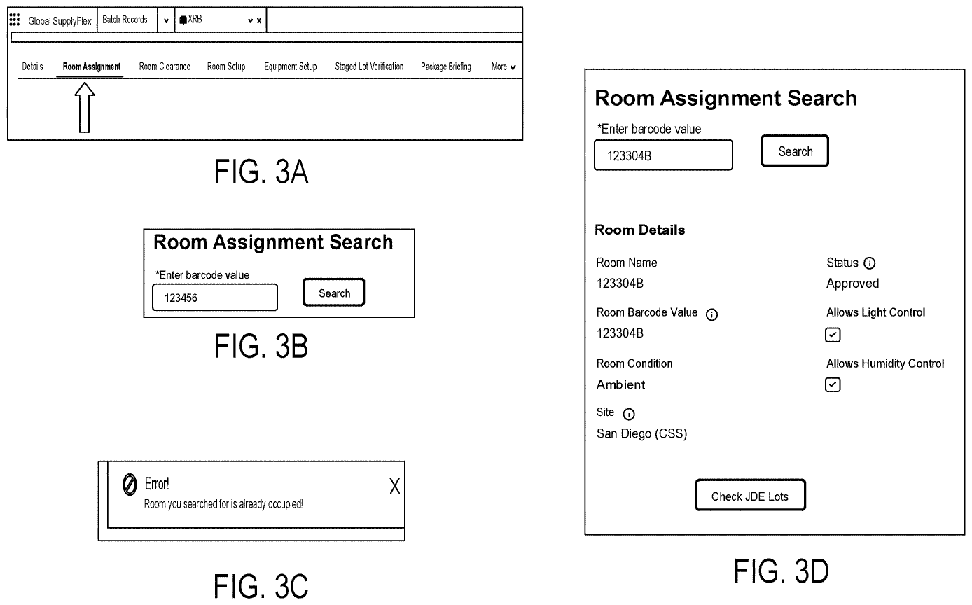

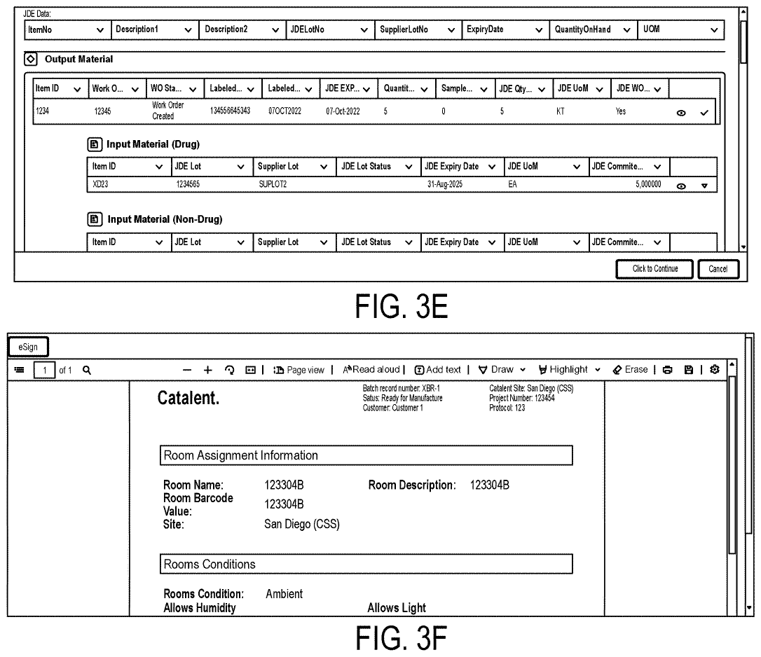

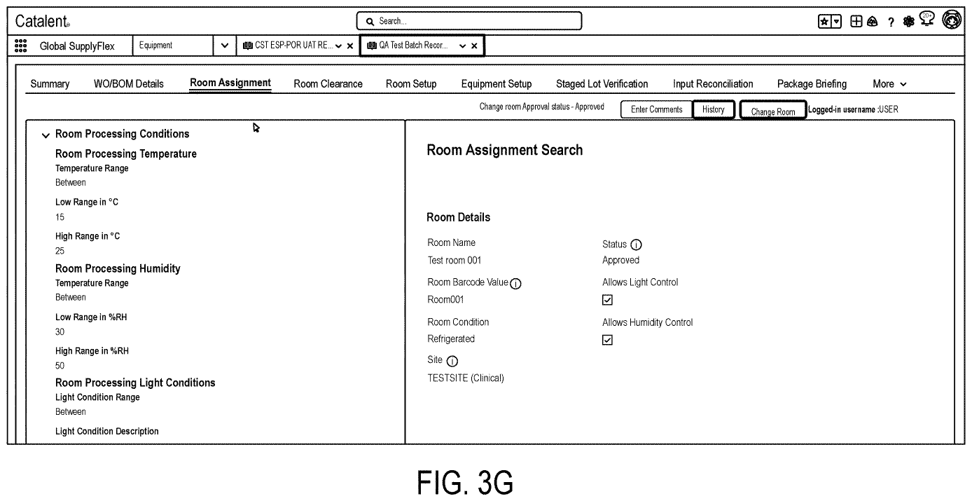

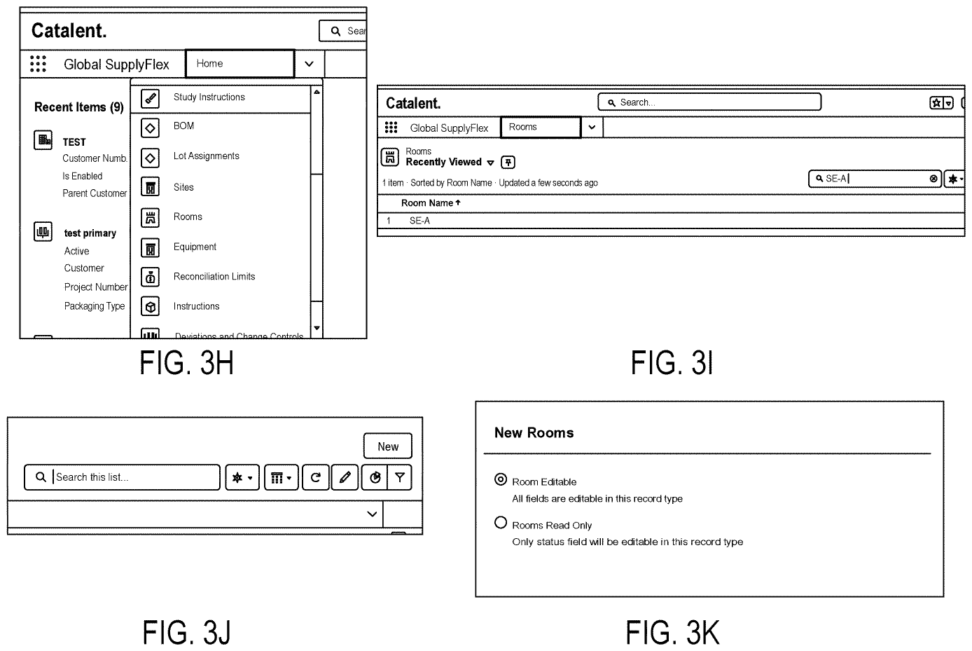

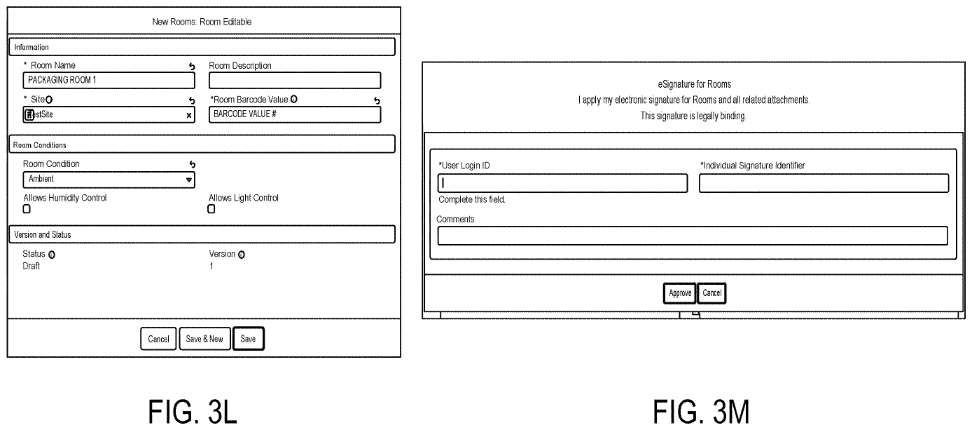

In one or more examples, the room assignment can be performed by one or more users. In some examples, the room assignment can be performed by users associated with a particular access permission (e.g., Team Lead). In one or more examples, the user completing the room assignment can select the Room Assignment tab within the batch record in execution selected at block 102, as shown in FIG. 3A. Referring to FIG. 3B, the Room Assignment tab may allow the user to check the availability of a room to be assigned to the batch record in execution. For instance, the system can receive a barcode value associated with a room to check the availability. In some examples, a user can physically scan a barcode located outside the packaging room at the packaging site. In some examples, the user can manually enter barcode associated with the packaging room. If the packaging room is currently assigned to another batch record in execution, an error message is displayed as shown in FIG. 3C. If the packaging room is available, the “Room Details” fields may be displayed below the “Enter barcode value” text input box, as shown in FIG. 3D. For each packaging room, one or more of the following “Room Details” fields may be displayed: “Room Name,” “Room Barcode Value,” “Room Condition,” “Site,” “Status,” “Allows Light Control,” and “Allows Humidity Control.” Additional fields may be included without departing from the scope of this disclosure.

Referring to FIG. 3D, the system may receive a user input indicating that the user has selected the “Check Lots” button. The system can then perform a room location lookup and display all materials currently in the packaging room. Additionally, in some examples, the system can display a pop-up window displaying all bill of materials (BOM) details for each output material and its corresponding input material(s) that are associated with the packaging room, as shown in FIG. 3E. The user can review the materials currently in the packaging room and compare the materials in the packaging room to the BOM details. If any material(s) displayed in the packaging room should not be present in the room during packaging of the batch record in execution, the user may select the “Cancel” button to exit the room assignment. The user may then clear the packaging room of any materials not required for the packaging job or chooses a different room for the packaging job. In some embodiments, a user may add equipment to a room, e.g., by physically introducing the equipment to the room and updating the information in the system. If all materials displayed in the packaging room can remain in the room during packaging, the user can select the “Click to Continue” button. Upon receiving the user selection of the “Click to Continue” button, the system can display an e-signature page for the user to complete, as shown in FIG. 3F. The user can confirm the room assignment and e-sign to complete the room assignment process. For instance, by receiving the user credentials, the system may attribute an e-signature to the user and indicate that the room assignment is approved. FIG. 3G illustrates an exemplary Room Assignment tab after the system has received the e-signature from the user competing the room assignment.

In one or more embodiments, prior to completing the Room Assignment on the system, the batch record in execution may already be associated with a packaging room on the site. In such embodiments, the room assignment may still be completed by the user to ensure that the room is not associated with another packaging job and/or that the room includes the appropriate equipment to complete the packaging job for the batch record in execution.

In some embodiments a packaging room may not be configured in the system. If the packaging room does not exist in the system, the system allows users to add a new packaging room to the master data. To add a packaging room, the system may receive a user selection of the “Home” dropdown menu on the user interface and further receive a selection of the “Rooms” option, as shown in FIG. 3H. Referring to FIGS. 31 and 3J, the system can present the Rooms tab, which includes a “New” button for adding a new room. Upon receiving the user's selection of the “New” button, the system presents a “New Rooms” window with user affordances corresponding to “Rooms Editable” and “Room Read Only” buttons, as shown in FIG. 3K. The system may then present a “New Rooms: Room Editable” window or “New Rooms: Room Read Only” window with user affordances for various fields, including, but not limited to, room name, site, room description, room barcode value, room condition, humidity control, and light control. If the user selects the “Room Read Only” button, the system may allow the user to edit these user affordances, as shown in FIG. 3L. If the user selects the “Room Read Only” button, the system may not allow the user to edit one or more fields associated with the packaging room. Upon receiving the user selection of the “Save” button on the window, the system can display an e-signature page for the user to complete, as shown in FIG. 3M. The user can then e-sign (e.g., enter their user credentials) to add the new room to the master data. The system may then allow the user to assign the packaging job to the new room as described above.

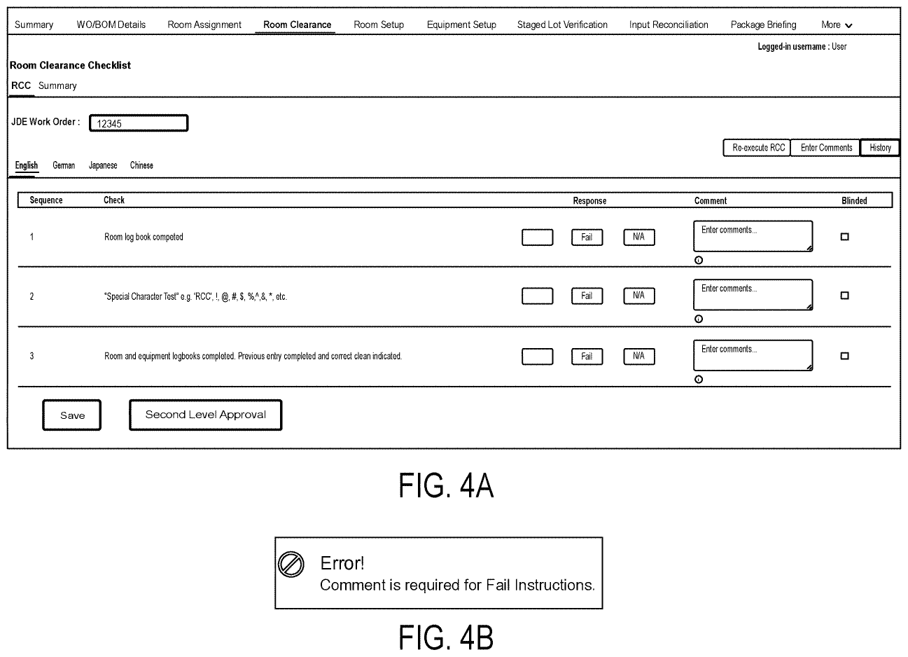

Referring back to block 106 of FIG. 1A, the system can present a Room Clearance tab including a user interface for a user to complete room clearance. In one or more embodiments, the room clearance is performed upon completion of the room assignment. FIG. 4A illustrates an exemplary user interface for electronically completing the room clearance process. As shown in FIG. 4A, the Room Clearance tab includes first section that includes a text input box that allows a user to enter a work order number, and a second section that provides a checklist. The checklist may correspond to instructions that a user should complete in order to clear the room and ensure the room is cleared to perform the packaging job.

In one or more embodiments, the system can receive an input from a user selecting the Room Clearance tab for the batch record in execution. The system may then receive an entry from the user in the text input box corresponding to the work order number for the batch record in execution. The system may then present the user with one or more steps associated with a checklist for the user to complete in order to clear the room (e.g., ready the room for packaging). As shown in FIG. 4A, the instructions may be presented in different languages depending on the packaging site where the user is located. The system can complete the “Room Clearance Checklist” by receiving a user selection of a response of “Pass,” “Fail,” or “N/A” for each step, and saving the responses. If one or more steps of the checklist has a response of “Fail,” the system can display an error message, as shown in FIG. 4B. The user may then follow a standard operating procedure to address the error(s). In order to complete the room clearance process, each step should have a response of “Pass” or “N/A.”

Once each of the steps meet these criteria (e.g., “Pass” or “N/A”), the system can receive approval by a first approver. In one or more embodiments, the first approver may have credentials associated with a particular access permission (e.g., Operator or Team Lead). The system can receive an e-signature of the first approver to indicate that the completed checklist has been approved. As used herein, receiving an e-signature from a user may correspond to receiving the user credentials (e.g., user login identifier and a unique signature identifier associated with the user). In some embodiments, the system is configured to receive additional approval by a second user before completing the room clearance. A second approval is performed by a second approver with a particular access permission (e.g., Team Lead or Ops QA) who independently performs second verification of room clearance according to the steps of the checklist. For instance, the system can receive a selection of the “Second Level Approval” user affordance once the second approver has confirmed that all steps of the room clearance tab meet the room clearance criteria. The system may then receive the second user's credentials for approval and the completion of the room clearance process.

In one or more embodiments, one or more devices at the packaging site may be used by the first and second users to execute the batch record process. In some embodiments a device may be used by multiple users to execute the batch record process. In such embodiments, although the system may be logged into a first user's account on the device, the e-signature and/or approval may be entered a second user. This is because the system attributes the e-signature to a user based on the credentials received during the e-sign process instead of automatically attributing an e-signature to the user that is currently signed into the system. This feature allows packaging teams to work smoothly and cohesively when using embodiments of the present disclosure. For example, multiple users can affix their e-signature to approve steps of the batch record execution process without having to sign off of the current account. Where multiple users are sharing a device to execute the batch record, permitting multiple users to enter their approval without having to switch accounts saves time and streamlines the packaging process. For instance, having a first user to e-sign, sign-out of the system and the second user sign in to approve a step is cumbersome and time consuming. Accordingly, by attributing the e-signature to a user based on credentials entered during the e-signature process as opposed to the account used to complete the packaging run, embodiments of the present disclosure provide and auditable approval trail (e.g., approvals that comply with relevant regulations) that saves time on the packaging floor and can contribute to a more efficient packaging run.

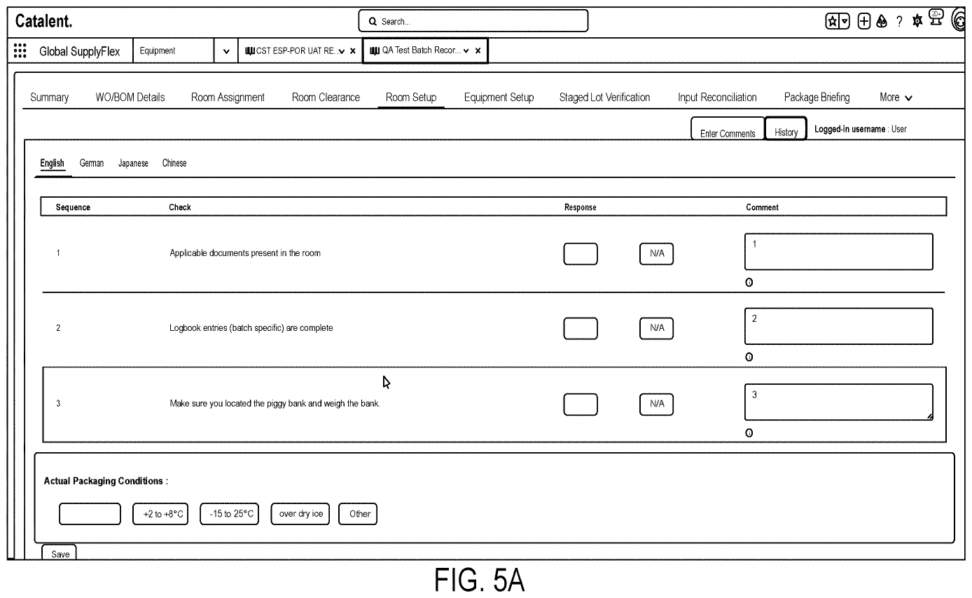

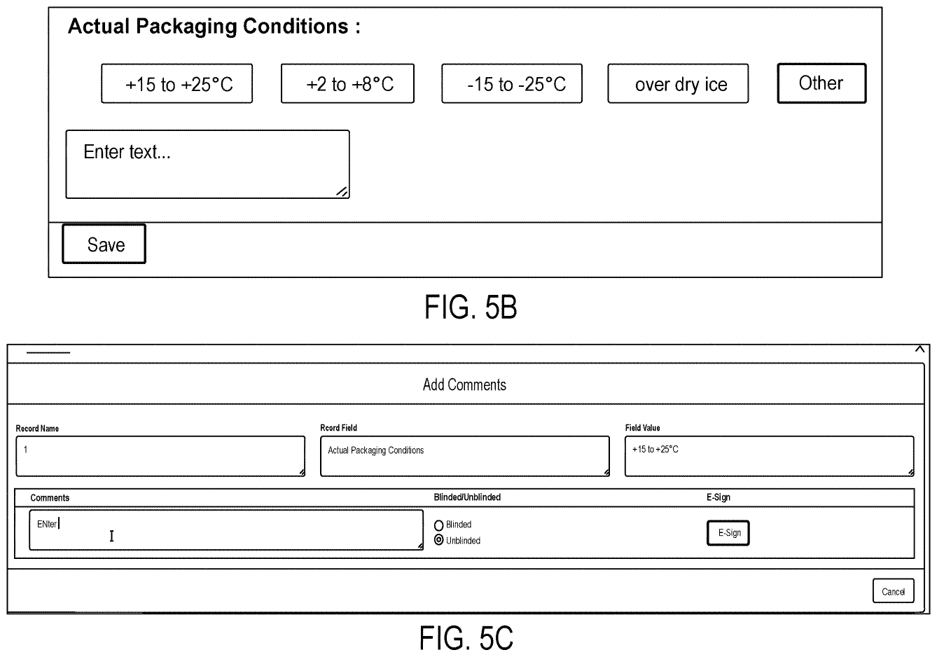

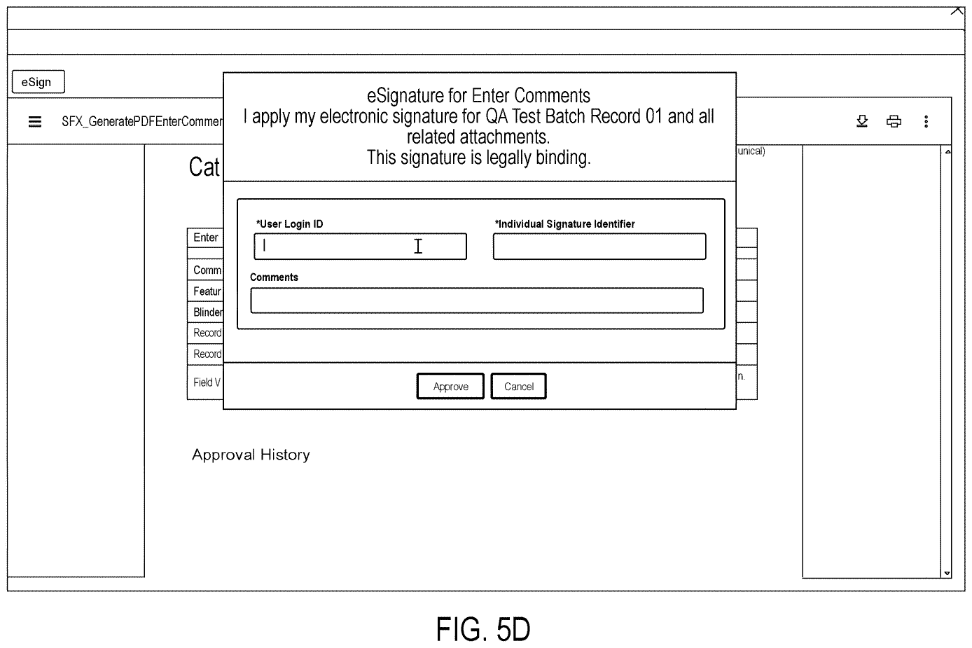

Referring back to block 108 of FIG. 1A, the system can present a Room Setup tab including a user interface for a user to complete room setup. In one or more embodiments, the room setup is performed upon completion of the room assignment and the room clearance. In one or more embodiments, the room setup process is completed once the room assignment process and the room clearance process have been completed. To initiate the room setup process, the system may receive an indication that a user has selected the Room Setup tab within the batch record in execution, as shown in FIG. 5A. The system may display a “Room Setup Checklist” and “Actual Packaging Conditions” on the Room Setup tab. The “Room Setup Checklist” provides the user with a checklist to confirm that the room is properly set up for packaging the batch record in execution. The “Actual Packaging Conditions” provides the user with an interface to enter information associated with the actual conditions of the room.

Regarding the “Room Setup Checklist,” the system may present the user with one or more steps and a corresponding option for the user to select a response associated with the step. As shown in FIG. 5A, the Room Setup tab may provide user affordances corresponding to “Pass” or “N/A” buttons for each step associated with the “Room Setup Checklist.” The steps listed in the “Room Setup Checklist” may include one or more of the following checking steps: “Color of the room status indicator outside the door has changed,” “Room is set up as per Batch Record temperature, humidity, and/or light conditions,” “Appropriate pressure difference between the room and the outside environment,” “Tables and chairs are set up for proper packaging operation,” “Room is set up as single piece flow,” “Room is in the correct status (e.g., cleaned for production),” “Special instructions (e.g., wearing PPE) have been implemented,” and “Batch-specific logbook entries have been completed.” In some embodiments more or less steps may be included in the “Room Setup Checklist.” As shown in the figure, in one or more embodiments, the “Room Setup Checklist” may be provided in one or more languages depending on the location of the packaging job. In one or more embodiments, the system can receive a comment from the user regarding the one or more steps of the “Room Setup Checklist.”

In one or more embodiments, as a part of the room setup process, the system may receive information entered by a user regarding the “Actual Packaging Conditions,” as shown in FIG. 5B. For instance, the “Actual Packaging Conditions” may include one or more user affordances for selecting a temperature range and/or temperature conditions. In some embodiments, the one or more user affordances may relate to humidity and/or lighting conditions. In one or more embodiments, the user interface may provide a text input box for the user to specify the actual packaging conditions.

Referring to FIG. 5C, the system may receive comments entered by the user regarding the room setup process. For instance, in one or more embodiments, the system may receive a selection of the “Enter Comments” user affordance shown in FIG. 5A. This may cause a Comments pop-up to appear, as shown in FIG. 5C. As shown in FIG. 5C, the Comments pop up can include a text input box where a user can enter comments associated with the room setup process and/or a user affordance where a user can indicate whether the setup is blinded or unblinded.

In one or more embodiments, each comment may be saved to the batch record in execution once the comment receives electronic sign-off. That is, the user entering the comment may affix their e-signature (e.g., by entering their user credentials) to the comment in order to save the comment to the batch record in execution. For instance, referring to FIG. 5C, the system receives the “Actual Packaging Conditions,” the blinding status, and/or comments entered by the user, the system may receive an indication that the user has selected the “E-sign” user affordance. The system may then present the user with one or more text input boxes to enter a user login ID, an individual signature identifier, and/or any additional comments, as shown in FIG. 5D. The system may receive the user credentials and/or comments as a part of to approve the comment. In one or more examples, the e-signature pop-up illustrated in FIG. 5D is exemplary and may be similar to other e-signature pop-ups described herein.

In one or more embodiments, although the system may be logged into a first user's account, the comment may be entered and signed off by a second user. This is because the system attributes the comment to a user based on the credentials received during the e-sign process instead of automatically attributing a comment to the user that is currently signed into the system. This feature allows packaging teams to work smoothly and cohesively when using embodiments of the present disclosure. For example, multiple users can affix their e-signature to comments and/or approval to steps of the batch record execution process without having to sign off of the current account. That is, in some examples, certain steps of the packaging process may solicit sign-off from multiple users (e.g., an operator and a Team Lead). Having a first user to e-sign, sign-out of the system and the second user sign in to approve a step is cumbersome and time consuming. Accordingly, by attributing the e-signature to a user based on credentials entered during the e-signature process as opposed to the account used to complete the packaging run, embodiments of the present disclosure provide and auditable approval trail that saves time on the packaging floor and can contribute to a more efficient packaging run.

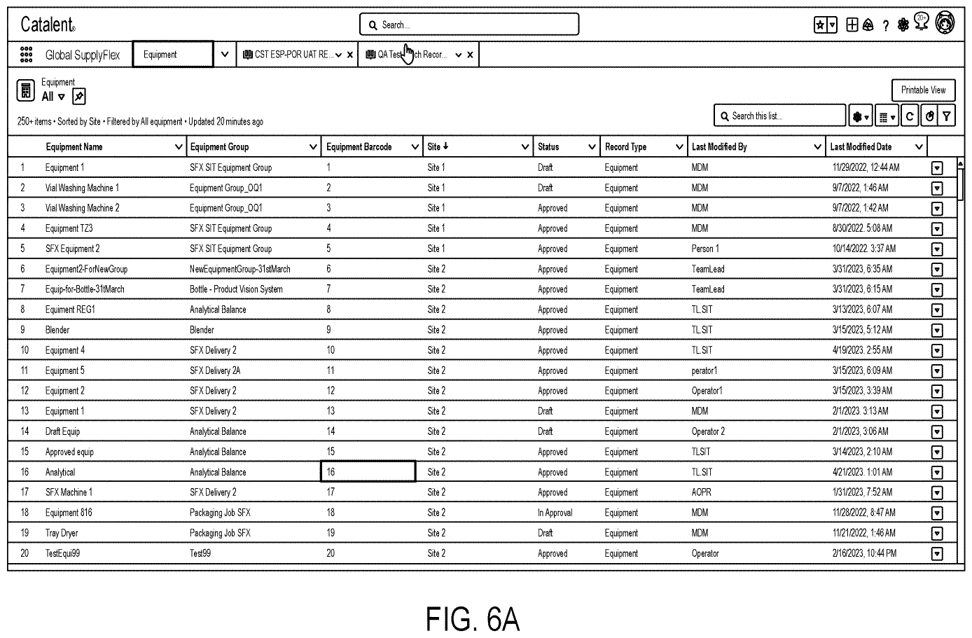

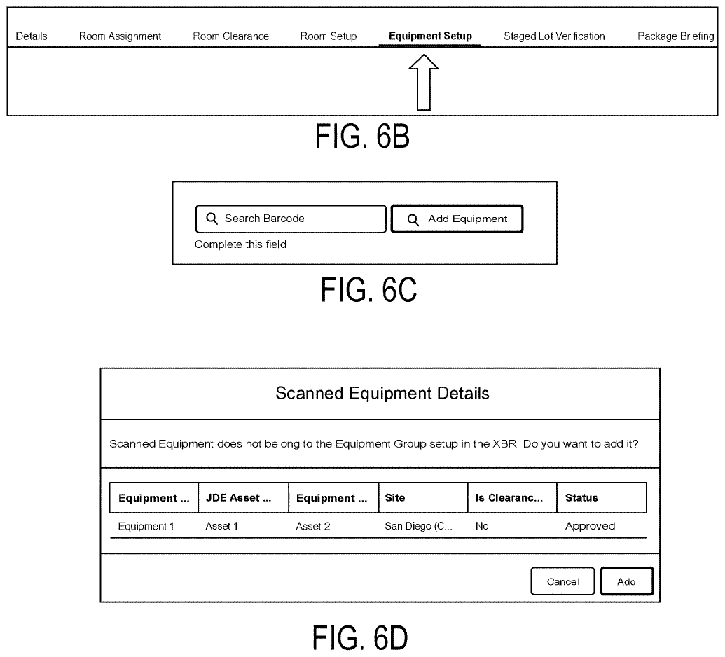

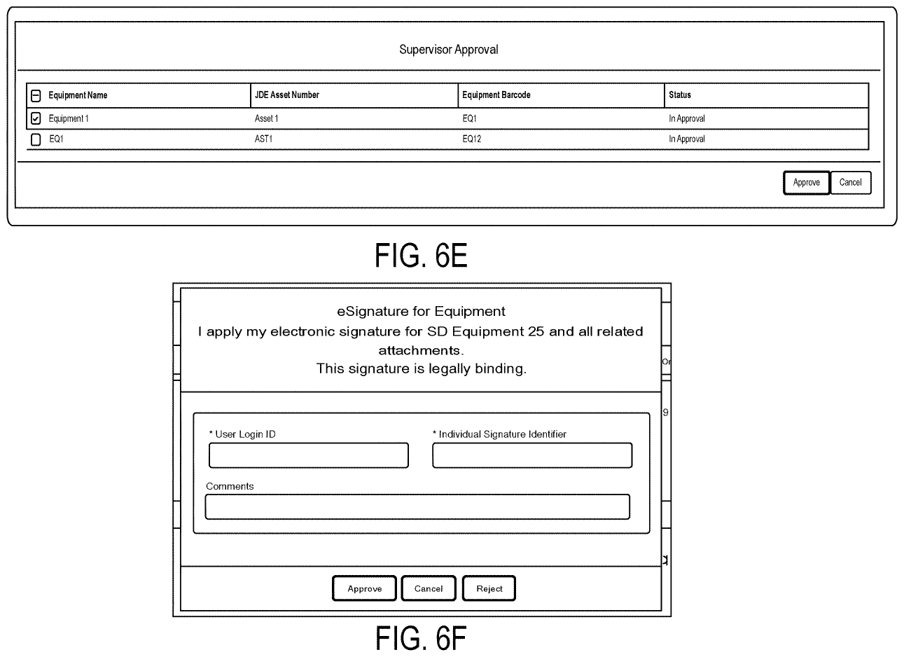

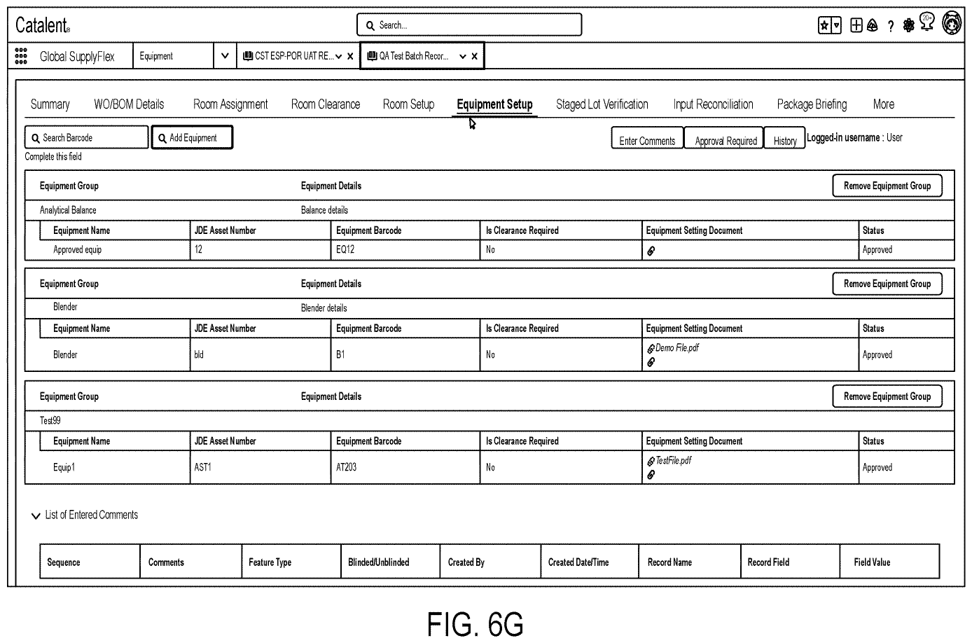

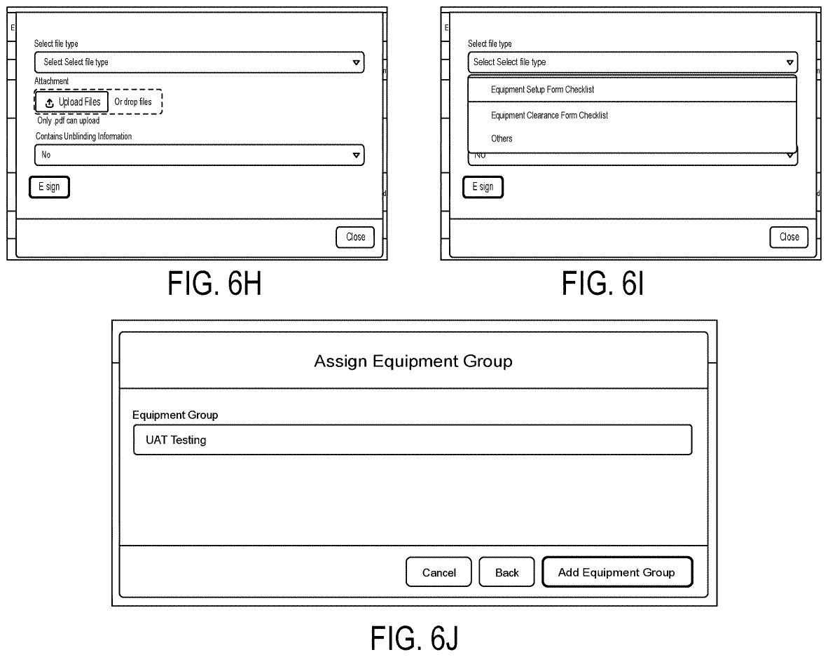

Referring back to block 110 of FIG. 1A, the system can present an Equipment Setup tab including a user interface for a user to complete equipment setup. In one or more embodiments, the equipment setup is performed upon completion of the room assignment, the room clearance, and room setup. The equipment setup may be used to add equipment to the batch record in execution. The system may include equipment in the master data that can be associated with the packaging run of a batch record in execution. As shown in FIG. 6A, fields associated with the equipment can include, but are not limited to, make and model, equipment name, asset number, the site where the equipment is utilized, approval status, and an equipment barcode.

Referring to FIG. 6B, to add equipment to the batch record in execution, the system can receive a user selection of the Equipment Setup tab of the user interface. The system can then receive a user input via a text input box corresponding to an “Add Equipment” user affordance, as shown in FIG. 6C. In some embodiments, the system may receive a barcode value by scanning and/or capturing an image of the equipment barcode of the desired equipment to be added (e.g., the equipment located in the assigned packaging room). For example, if the device used by the user comprises a camera, the device can capture an image of a barcode disposed on the equipment to identify the equipment in the system. In some embodiments, an error message may appear if the scanned equipment is not appropriately registered on the platform and/or not associated with the batch record in execution. In one or more embodiments, if the scanned equipment is not associated with the batch record in execution, the system may permit a user to manually add the equipment to the batch record in execution. In one or more embodiments, a user may also remove equipment associated with a batch record in execution (e.g., if the equipment was not available and/or not used in the packaging run).

If the scanned equipment is registered, the system can display a “Scanned Equipment Details” pop-up window, as shown in FIG. 6D. The system may then receive an indication that the user has selected the “Add” button to confirm the addition of the equipment to the batch record in execution. The system may further receive an e-signature from the user as described above. In some embodiments, the addition of equipment may be approved by a second user with a predetermined access status (e.g., a team lead). In one or more examples, the second user may select an “Approval Required” user affordance, which causes the system to display a “Supervisor Approval” pop-up window as shown in FIG. 6E. The system may display an e-signature pop-up window, as shown in FIG. 6F, and receive the second user's credentials to complete the equipment setup process. As discussed above, in one or more embodiments, the second user may e-sign using the same electronic device as the first user without logging off of the account of the first user.

In one or more embodiments, each piece of equipment to be used in the packaging run for the batch record in execution may be added in the manner described above. Upon completion of an equipment setup, a corresponding equipment setup document can be added to a list of approved equipment associated with the batch record in execution. An exemplary list of equipment added to a batch record is shown in FIG. 6G.

In one or more examples, the system may permit a user to attach one or more files to a piece of equipment during the equipment setup. In such examples, the system can receive a user selection of an attachment icon under the “Equipment Setting” column. In response to this selection, the system can open a pop-up window for file attachments, as shown in FIG. 6H. As shown in FIG. 6I, the system can receive a user selection of the appropriate file type, receive the uploaded file, receive a selection of “Yes” or “No” for whether the file “Contains Unblinding Information,” and receive an e-signature from the user. In one or more embodiments, the file may be saved locally on the electronic device or accessed via the cloud. The system may then save the equipment setup document to the corresponding equipment on the batch record in execution.

In one or more embodiments, each piece of equipment may be assigned to an “Equipment Group.” The equipment group can refer to equipment of a certain type, e.g., “Induction Sealer.” An “Equipment Group” can be added during the creation of the executable batch record and can be displayed on the Equipment Setup tab in the user interface of the batch record in execution. If an “Equipment Group” added during the creation of the executable batch record is determined to not be necessary to complete the batch record in execution, the equipment group can be removed upon receiving two e-signatures from two approvers with appropriate access permissions (e.g., as discussed above-receiving approval from an operator and a supervisor). If an “Equipment Group” is not added during the creation of the executable batch record, but it is determined that a specific equipment group should be used to complete the packaging job for the batch record in execution, the specific equipment group can be added during execution of the batch record upon receiving two e-signatures from two approvers with appropriate access permissions. FIG. 6J illustrates an exemplary pop-up window for adding an “Equipment Group” to the batch record in execution.

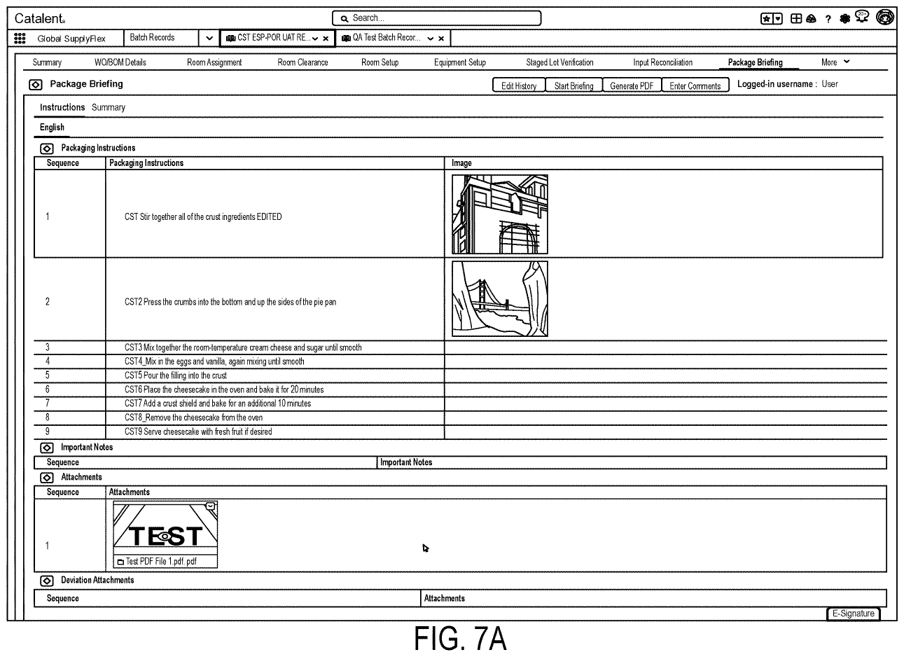

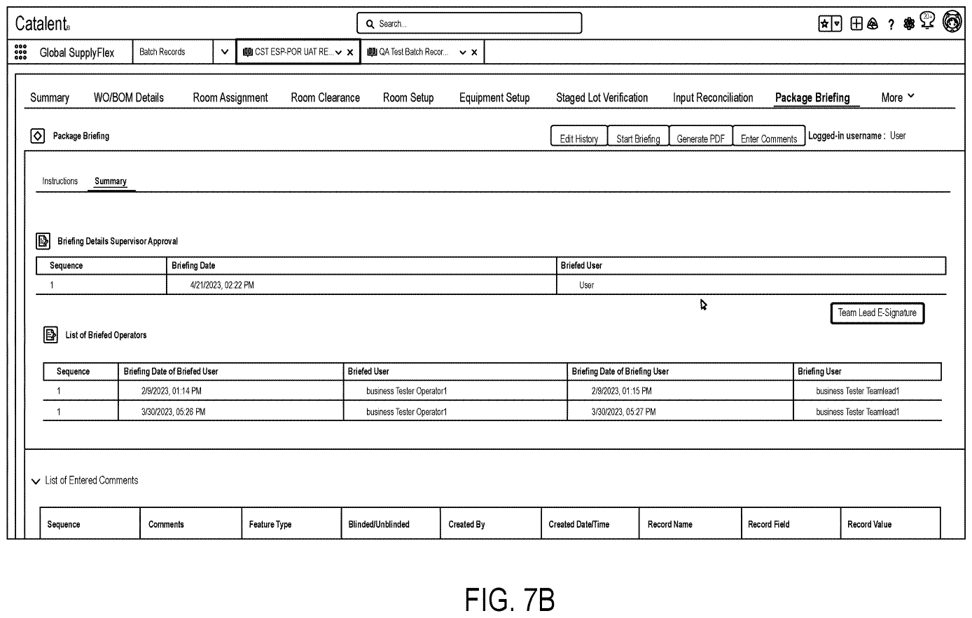

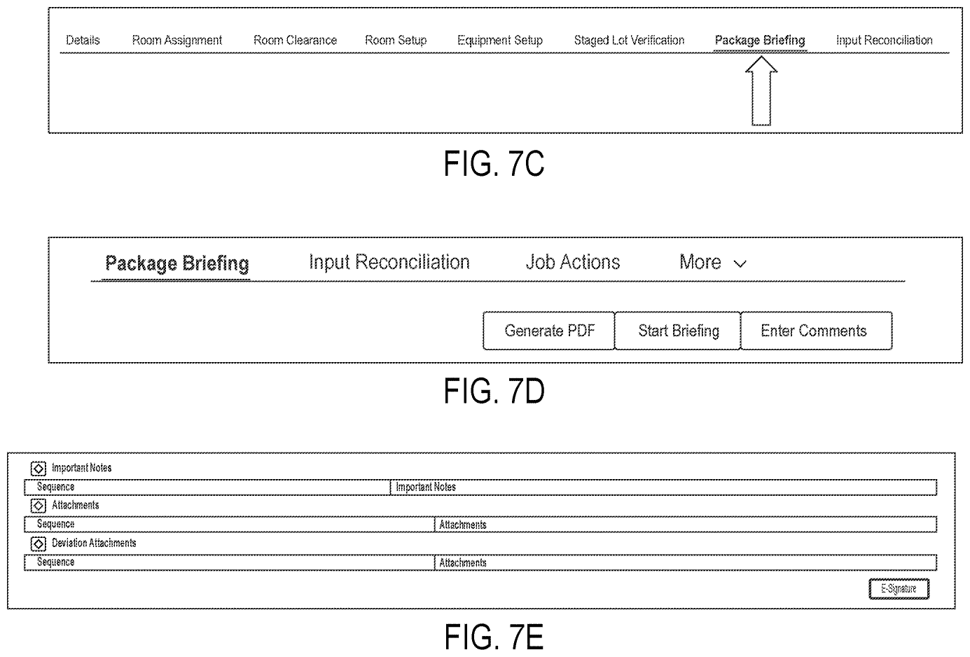

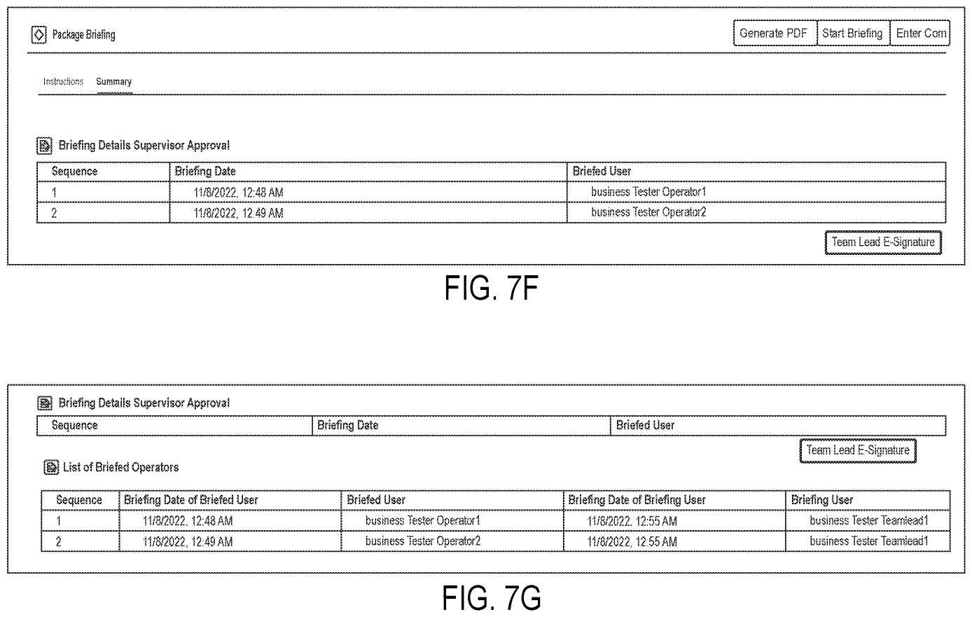

Referring back to block 112 of FIG. 1A, the system can present a Package Briefing tab including a user interface that includes information for a first user (e.g., operator) to review and/or be briefed by a second user (e.g., supervisor or team lead) on the packaging process. In some examples, one or more operators may be presented with the Package Briefing tab and complete the briefing process. In such examples, a user with a designated access permission (e.g., Team Lead) may sign-off or otherwise approve that the one or more operators completed the package briefing. Referring to FIGS. 7A and 7B, the Package Briefing Tab may include an Instructions page and a Summary page. The Instructions page may comprise a plurality of packaging instructions associated with the batch record in execution. The summary page may comprise a summary comprising a list of which users (e.g., operators) were briefed and the date and/or time of the briefing.

In order to complete the package briefing, the system may receive an indication that a user has selected the Package Briefing tab for the batch record in execution, as shown in FIG. 7C. Referring to FIG. 7D, the Package Briefing tab may allow the user to select a user affordance (e.g., “Start Briefing) to initiate the package briefing process for the batch record in execution. In one or more examples, the system can display various fields that the second user (e.g., team lead) may review with the users (e.g., operators) being briefed as a part of the package briefing process. FIG. 7E illustrates some exemplary fields. The fields may, include, but are not limited to, sequence, packaging instructions, packaging conditions requirements, work order and/or BOM details, important notes, and other attachments. Additional fields may be included without departing from the scope of this disclosure. After the second user (e.g., team lead) has briefed the one or more first users (e.g., operators), each of the first users can e-sign indicating that they have completed the briefing.

Once one or more first users (e.g., operators) have completed the review of the briefing for the batch record in execution and e-signed, the second user (e.g., team lead) can e-sign by selecting the “Team Lead E-Signature” button on the Summary section, as shown in FIG. 7F. This approval by the supervising team lead is used to confirm that the one or more operators have completed the package briefing. Referring to FIG. 7G, upon receiving the e-signature from the team lead that one or more operators have completed the package briefing, the system can display a list of users that have completed the package briefing under the Summary section of the Package Briefing tab.

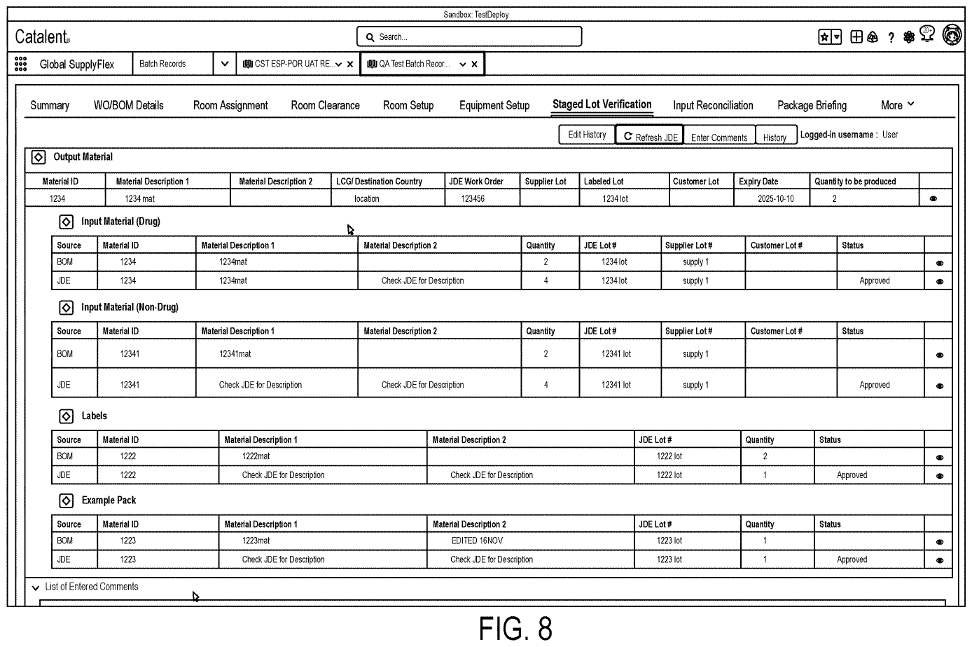

Referring back to block 114 of FIG. 1A, the system can present a Staged Lot Verification tab including a user interface for a user to verify whether materials staged in the packaging room match the bill of materials (BOM) associated with the batch record in execution. In one or more examples, the system can receive a selection by a user (e.g., operator) of the Staged Lot Verification tab within the batch record in execution selected at block 102, as shown in FIG. 8. Once input materials are staged into the packaging room, the user can select the “Refresh” user affordance highlighted in FIG. 8. A selection of the “Refresh” button may cause the system to obtain information from an external resource, such as, but not limited to JD Edwards. In some embodiments, the external resource may comprise information related to the bill of materials and available and/or assigned supply lots to be used for the packaging run. If the staged input materials match the BOM, no further actions may be required, and the staged input materials can be displayed in the batch record in execution with an “Approved” status. If the staged input materials do not match the BOM, the system can display an option to either “Accept” the mismatched input material(s) (i.e., add the input material(s) to the batch record in execution) or “Reject” the mismatched input material(s). For instance, the system may flag the material as a mismatch if the item number, supplier lot, and/or the customer lot number do not match those listed in the BOM. For each input material, selecting either “Accept” or “Reject” may require an e-signature from a user with a particular access permission (e.g., team lead).

Once each input material has been added to the batch record in execution by the user completing the staged lot verification, the system can receive approval by a first approver. In one or more embodiments, the first approver may have credentials associated with a particular access permission (e.g., team lead). The system can receive an e-signature of the first approver to confirm the status of each input material (e.g., “Approved,” “Accepted,” or “Rejected”). In some embodiments, the system is configured to receive additional approval by a second user before completing the staged lot verification. A second approval can be performed by a second approver with a particular access permission (e.g., quality assurance operator) who may independently confirm the status of each input material. The system can receive an e-signature of the second approver to complete the staged lot verification process.

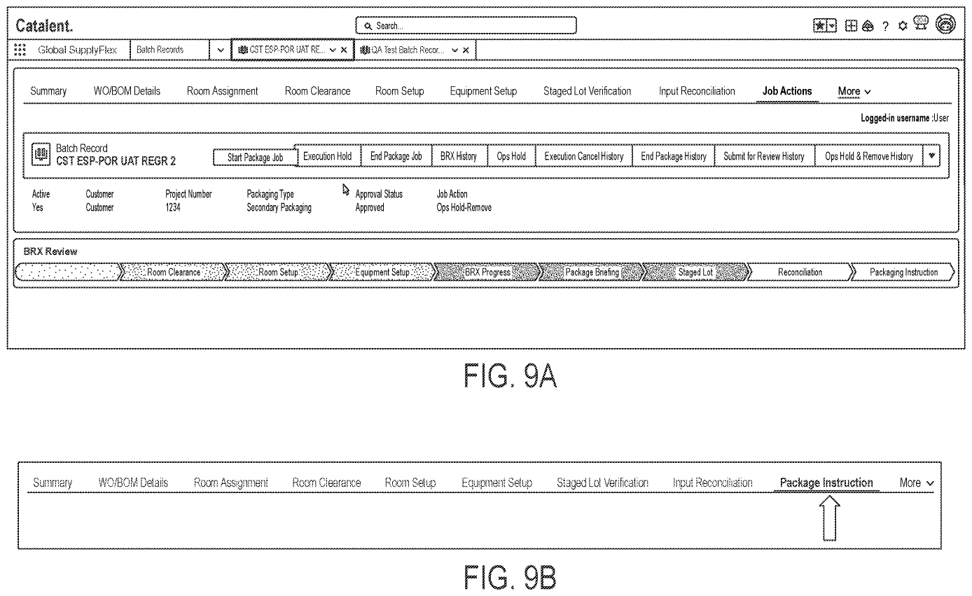

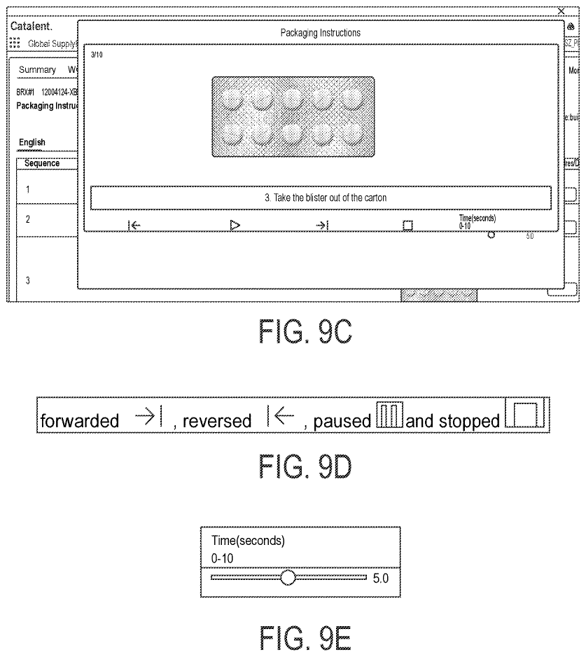

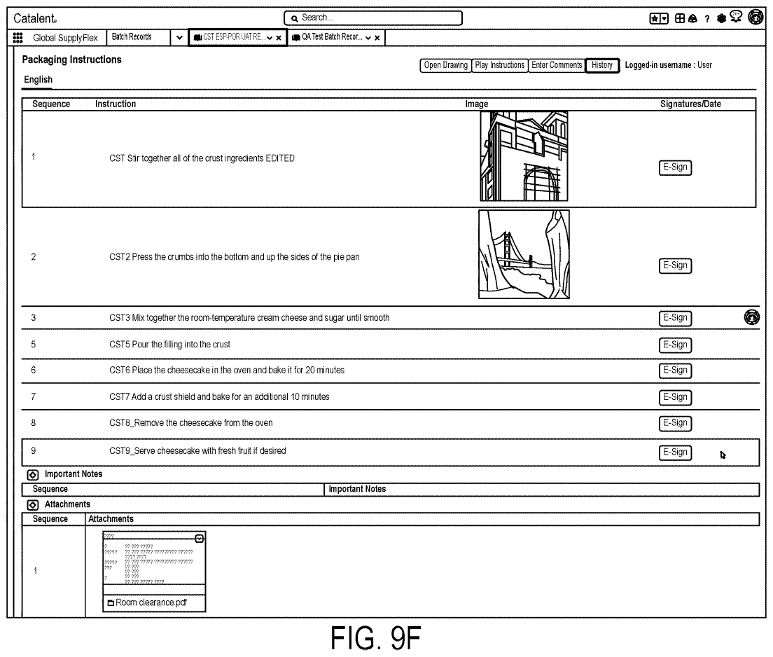

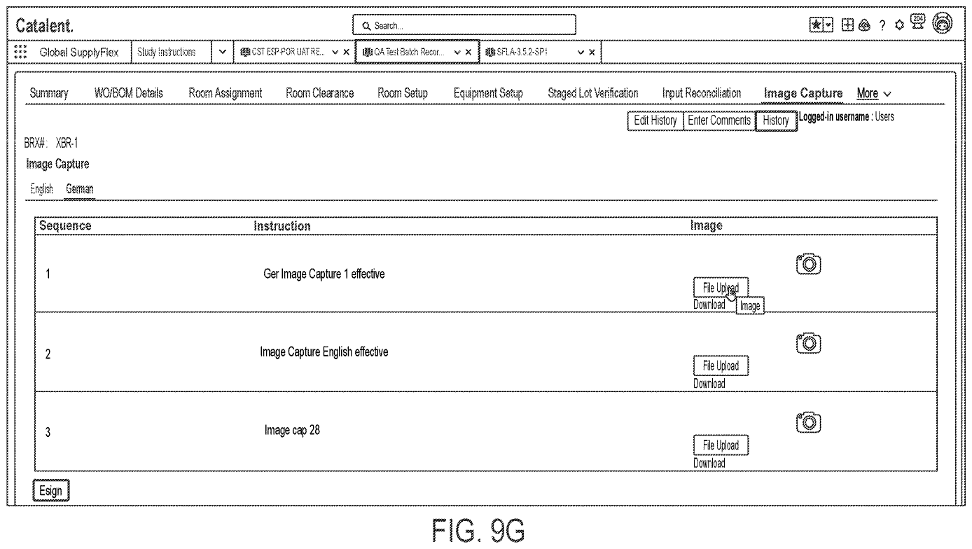

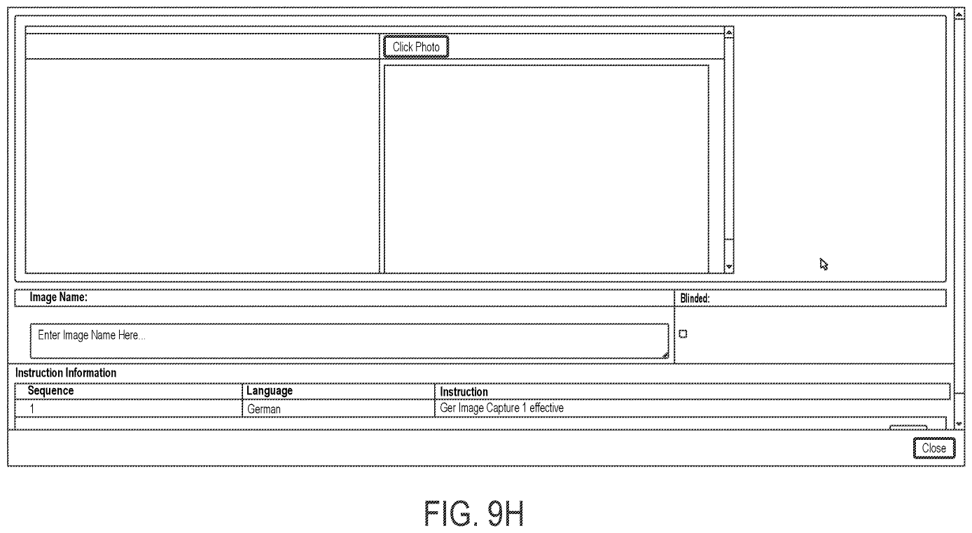

Referring back to block 116 of FIG. 1A, the system can start a packaging job and display packaging instructions. For instance, the system can present a Job Actions tab including a user interface for a user to perform actions related to a packaging job, e.g., starting the packaging job. In some examples, the packaging job can be started by users with a designated access permission (e.g., team lead). FIG. 9A illustrates an exemplary user interface for starting the packaging process. In one or more examples, the user starting the packaging job can select the “Start Packaging” button located on the Job Actions tab. The system may prompt a user may to e-sign to confirm that the packaging job has started. In some embodiments, if the access permissions of the user who entered the e-signature do not match the designated access permission, the system may not approve start of the packaging job.

At any point after starting the packaging job and before ending the packaging job, the batch record in execution may be placed on Execution Hold if there is a suspected or known quality event that may impact the safety, quality, identity, potency, and/or purity (SQuIPP) of the materials. If a batch record in execution is placed on Execution Hold, it will become read-only until the Execution Hold is removed. In some examples, the Execution Hold may be placed by users with a first designated access permission (e.g., team lead or quality assurance operations leader). In one or more examples, the user placing the Execution Hold can select the “Execution Hold” button on the Job Actions tab, as shown in FIG. 9A. The system may receive an e-signature from the user as a confirmation that the batch record in execution should be placed on Execution Hold. The system may then change the job status of the packaging job from “In Execution” to “Execution Hold.” In some examples, the Execution Hold may be removed by users with a second designated access permission (e.g., quality assurance operator). In one or more examples, to remove an execution hold, the system can receive a user selection of the “Remove Execution Hold” button on the Job Actions tab. The system may then receive an e-signature from the user to confirm that the batch record in execution should be removed from the Execution Hold. The system may change the job status of the packaging job from “Execution Hold” to “In Execution.” In some embodiments, if the access permissions of the user who entered the e-signature do not match the designated access permission, the system may not approve the execution hold and/or remove the execution hold of the packaging job.

At any point after starting the packaging job and before ending the packaging job, the batch record in execution may be placed on Operational Hold if there is no active packaging occurring (e.g., between shifts, at the end of the day, or during breaks/lunch). In some examples, the Operational Hold may be placed and removed by users with a designated access permission (e.g., team lead). In one or more examples, the system can receive a user selection of the “Ops Hold” button on the Job Actions tab shown in FIG. 9A to put the packaging run operation on hold. In one or embodiments, this operational hold may be based on a timer displayed on the Job Actions tab (e.g., not shown). The system may prompt the user to e-sign to confirm that the batch record in execution should be placed on Operational Hold. The job status of the packaging job may change from “In Execution” to “Ops Hold.” In one or more examples, the system can receive a user selection of the “Remove Ops Hold” button on the Job Actions tab to remove the Operational Hold. The user may then e-sign to confirm that the batch record in execution should be removed from the Operational Hold. The job status of the packaging job may change from “Ops Hold” to “In Execution.” In some embodiments, if the access permissions of the user who entered the e-signature do not match the designated access permission, the system may not approve the operational hold and/or remove the operational hold of the packaging job.

Upon receiving an indication that the packaging job has started, the system may be configured to display packaging instructions to a user. For instance, the system can present a Packaging Instructions tab including a user interface for a user to display and execute the packaging instructions. In some examples, the packaging instructions can be selected for display by users with a designated access permission (e.g., operator or team lead). In one or more examples, a user (e.g., operator) who has completed the package briefing will be able to view the packaging instructions. If a user has not completed the packaging briefing, the user may be prompted to complete the packaging briefing before the system will display the packaging instructions.

In one or more examples, the system can receive a selection, by the user, of the Packaging Instructions tab for the batch record in execution, as shown in FIG. 9B. In some instances, as shown in FIG. 9C, the system may display the packaging instructions on screen as a slideshow, wherein each instruction and the respective image (if applicable) is shown one by one. The packaging instructions can be forwarded, reversed, paused, and stopped by selecting the applicable icons, as shown in FIG. 9D. In some embodiments, the system may display each instruction for a set amount of time (e.g., five seconds). However, the system can increase or decrease this display duration by receiving a user input adjusting the time bar shown in FIG. 9E. In such embodiments, as a step of the packaging instructions is displayed, the user may execute the step according to the instructions. The system may then receive the user's e-signature to confirm completion of the step. The e-signature may contain fields including, but not limited to, the user's name and the timestamp of the e-signature. In one or more embodiments, the instructions may be displayed as a list form, as shown in FIG. 9F. In such embodiments, the system can allow the user to scroll through each of the steps and receive an e-signature from the user upon the user completing corresponding step.

In one or more embodiments, as a part of executing the packaging instructions, the system may prompt the users(s) executing the packaging job to capture images of one or more steps of the packaging job during execution in the packaging room. For instance, one or more steps of the packaging instructions may include a prompt requesting the user capture an image to document that the corresponding step has been completed. In such embodiments, the system may capture or receive images of the packaging job after starting the packaging job (e.g., as a part of block 116 of FIG. 1A).

As shown in FIG. 9G, the system can present an Image Capture tab including a user interface for a user to capture images. As shown in the figure, packaging instructions may be displayed in sequence on the Image Capture tab. The system may provide the user with options to capture and attach images associated with each packaging instruction. In some examples, the image capture can be performed by users with a designated access permission (e.g., operator, team lead, or quality assurance operators). The system may capture images on a local device (e.g., electronic device used by the users fulfilling the packaging job) or camera associated with the system. In an example, the system may present the user with a “Camera” button and receive images captured by the user from the local device or camera. For instance, the user may view the packaging instructions on a tablet or mobile device. This same tablet or mobile device may be used to capture the images. In one or more embodiments, the system may receive images uploaded from an external device or camera. In an example, the system may present the user with a “File Upload” button and receive uploaded images captured on an external device or camera.

FIG. 9H illustrates an exemplary user interface that allows the user to select a particular image to be attached to a particular packaging instruction. If relevant images associated with each relevant packaging instruction have been added, the user can review all images against each instruction and select the “E-Sign” button. Upon receiving the user selection of the “E-Sign” button, the system can display an e-signature page for the user to complete. The user can then confirm the image capture and provide the system with their credentials to complete the e-signature. In an example, after receiving an e-signature from a first user, the system can display another e-signature page for a second user to complete. The second user can then independently review the captured images against each instruction, confirm the image capture, and provide the system with their credentials to complete the e-signature. In one or more embodiments, the system may obtain at least two signatures from users having different designated access permissions to save the images to the packaging instructions.

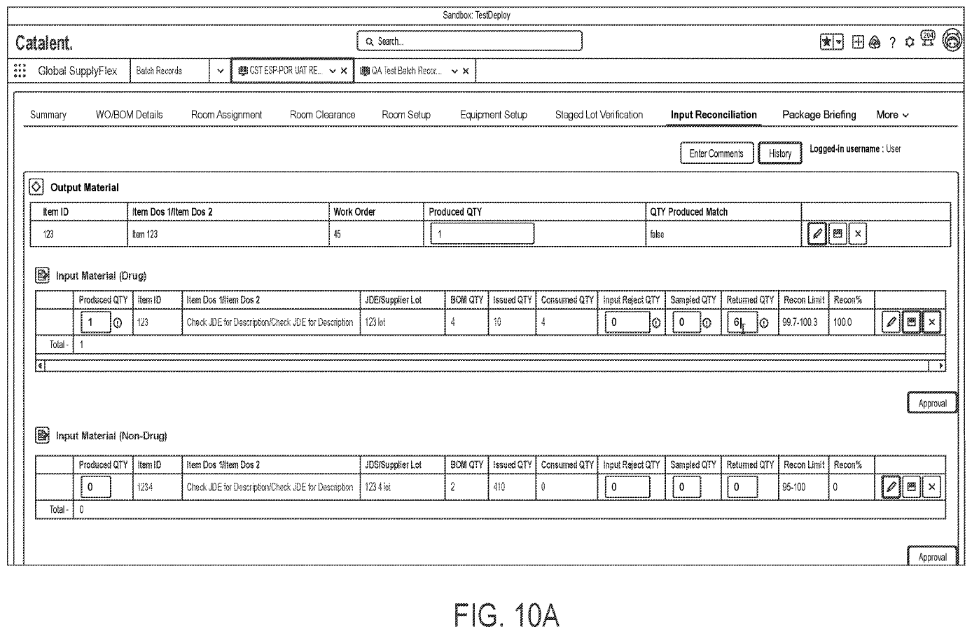

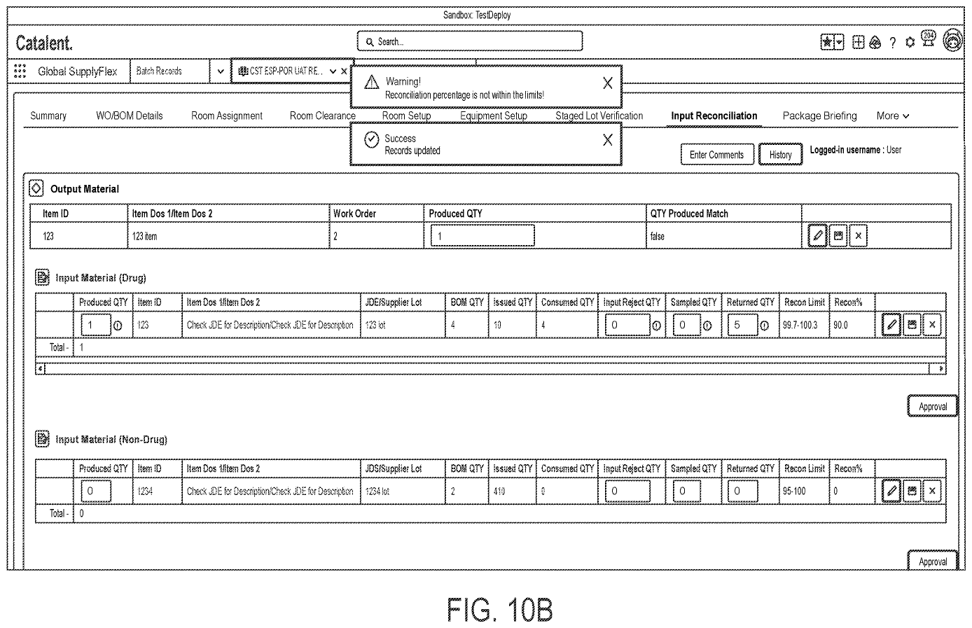

Referring back to block 118 of FIG. 1A, the system can present an Input Reconciliation tab including a user interface for a user to verify whether quantities of input materials are within their respective reconciliation limits. FIG. 10A illustrates an exemplary Input Reconciliation tab and associated user interface for electronically completing the input reconciliation process. As shown in the figure, materials staged during the Staged Lot Verification process can be displayed in the Input Reconciliation tab upon approval of the materials at step 114. Input materials are grouped under their respective output material. In one or more examples, one or more of the following input material fields may be displayed for each input material, as shown in Table 2:

TABLE 2

| Input Material | |
|---|---|
| Fields | Data Type |
| Produced QTY | Total output quantity produced at the time of |
| | Reconciliation. |
| BOM QTY | Quantity is pulled from the XBR BOM. |
| Issued QTY | Quantity is pulled from the respective staged |
| | material from Staged Lot Verification tab. |
| Consumed QTY | Quantity will be calculated based on the entry of the |
| | Produced QTY for the respective output material |
| | multiplied by the BOM QTY of the input material. |
| Input Reject | This is a user entry; this will be the number of |
| QTY | rejects produced for that given input material. |
| Sample QTY | This is a user entry; this will be the consumed |
| | quantity of that given input material for samples. |
| Returned QTY | This is a user entry; this will be the quantity |
| | returned from the staged quantity for that given |
| | input material. |
| Recon Limit | This a predefined value from the XBR. This will |
| | display the lower and upper reconciliation limit for |
| | the given input material. |
| Recon % | This is a calculated value based on the output |
| | material produced quantity, the entry of input, |
| | sampled, returned quantities entered by the user and |
| | the staged quantity. |
| | (Consumed QTY + Input Reject QTY + Sample |
| | QTY + Returned QTY)/(Staged QTY) * 100 = |
| | Recon % Calculated |

In one or more examples, one or more values associated with the bill of materials may be automatically populated on the Input Reconciliation tab based on information stored at the external resource (e.g., JD Edwards). For instance, these values may include, but are not limited to the BOM QTY, Issued QTY, and Consumed QTY. In such examples, if a user determines that one of these values associated with the bill of materials in incorrect, the user can update the values by visiting the external resource. For instance, the system can navigate and/or open a pop-up window corresponding to the external resource for the user to update these values.

In one or more examples, after a user has produced a quantity of output materials based on the packaging instructions (e.g., packaged one or more kits based on the packaging instructions), the user may use the Input Reconciliation tab of the system to verify that the quantity of packaged materials is within a predetermined reconciliation target. For instance, the system can receive an input that a user has selected the Input Reconciliation tab. The system can display a user interface as shown in FIG. 10A corresponding to the Input Reconciliation tab. At the Input Reconciliation user interface, the system can receive an indication that the user has selected the edit icon for the output material. The system can receive a data entry from the user for the “Produced QTY,” and save the entry. The system may receive data entries for each of the “Input Reject QTY,” “Sample QTY,” and “Returned QTY,” and save the entries. In one or more examples, the system may automatically calculate the “Recon %” (e.g., corresponding to the calculated reconciliation value) and display this value in the appropriate field.

If the if the calculated reconciliation value is within a predetermined reconciliation limit, the calculated reconciliation value is displayed in green. For example, referring to FIG. 10A, this input material has a BOM QTY of 4, an Issued QTY of 10, and a consumed QTY of 4. As shown in the figure, the system has received a Returned QTY value of 6 from the user. This provides a calculated reconciliation value (e.g., Recon %) of 100. The calculated reconciliation value appears as green text because this value is between the predetermined reconciliation limit of (99.7-100.3). In one or more examples, the reconciliation limit (e.g., reconciliation target) may correspond to a reconciliation target determined as a part of the executable batch record.

If the calculated reconciliation value is outside of the reconciliation limit, the calculated reconciliation value is displayed in red. For instance, referring to FIG. 10B, the input material has a BOM QTY of 4, an Issued QTY of 10, and a consumed QTY of 4. As shown in the figure, the system has received a Returned QTY value of 5 from the user. This provides a calculated reconciliation value (e.g., Recon %) of 90. Because the calculated reconciliation value is outside the reconciliation limit of 99.7-100.3, the calculated reconciliation value may be displayed in red and/or the system may display a warning pop-up banner indicating that the “Reconciliation percentage is not within the limits.”

The system may receive data entries for each of the input materials displayed on the Input Reconciliation tab (e.g., based on the bill of materials). In one or more examples, reconciliation is performed for each staged lot. For example, if three different staged lots are used for the same input material, the user interface will present separate input material reconciliation entries for each of the staged lots. This enables the reconciliation to be performed on a basis that is consistent with the inventory records stored by the external resource, which are organized by lot. The system allows input reconciliation to be completed more than once. Each reconciliation is captured in the reconciliation history.

Once input reconciliation has been calculated by the system, the system can receive a first approval from a first approver with a first access permission (e.g., an operator or a quality assurance operator). For instance, the first approver can review the reconciliation and the system can receive a user selection corresponding to the “Approval” button. In response to a user selection of the “Approval” button, the system can present an e-signature pop-up window for the first user to review and sign. Upon completion of the first approval, the system can display a “Second Approval Pending” message. In some embodiments, the respective input material may not be edited until a second approval is performed. The second approval may be performed by a second approver with an operator or quality assurance operator role. The second approver can review the reconciliation, and the system can receive the second user's selection of the “Approval” button. In response to the second user's selection of the “Approval” button, the system can present an e-signature pop-up for the second user to review and sign. In one or more examples, the system may request additional approval from users with a second access permission (e.g., from a team lead) if the calculated reconciliation value is outside the predetermined reconciliation limits.

The e-signature process for having multiple users approve and/or sign may be substantially similar to the e-signature process described above. For example, in response to receiving a selection of the “Approval” button from a user, the system may present the user with one or more text input boxes to enter a user login ID, an individual signature identifier, and/or any additional comments. In one or more embodiments, although the system may be logged into a first user's account, approval and/or e-signature may be attributed to a second user. This is because the system attributes the approval to a user based on the credentials received during the e-sign process instead of automatically attributing a comment to the user that is currently signed into the system. This feature allows packaging teams to work smoothly and cohesively when using embodiments of the present disclosure. For example, multiple users can affix their e-signature to approve steps of the batch record execution process in quick succession without having to sign off of the current account. For example, as is the case with approval for reconciliation limits, the system may request approval from more than one user. Having a first user to e-sign, sign-out of the system and the second user sign in to approve the reconciliation is cumbersome and time consuming. Accordingly, by attributing the e-signature to a user based on credentials entered during the e-signature process as opposed to the account used to complete the packaging run, embodiments of the present disclosure provide an auditable approval trail that saves time on the packaging floor and can contribute to a more efficient packaging run.

Referring back to block 120 of FIG. 1B, the system can receive an indication from a user to end the packaging job. For instance, the system can receive a user input corresponding to a selection of the Job Actions tab. Referring back to FIG. 9A, at the Job Actions tab, the system can present a user interface for a user to perform actions related to a packaging job, e.g., ending the packaging job. In some examples, the packaging job can be ended by users with a designated access permission (e.g., team lead). In one or more examples, the user ending the packaging job can select the “End Packaging” button located on the user interface corresponding to the Job Actions tab. In some embodiments, the system may prompt one or more users to e-sign to approve ending packaging job.

Referring back to block 122 of FIG. 1B, the system can produce an electronic executed batch record (EBR) for review. In one or more examples, following reconciliation and/or ending the packaging job, the system can convert the batch record in execution (BRX) to an electronic executed batch record (EBR). The executed batch record may reflect one or more comments, notes, updates, and/or actions taken with respect to the batch record during execution (e.g., the packaging process).

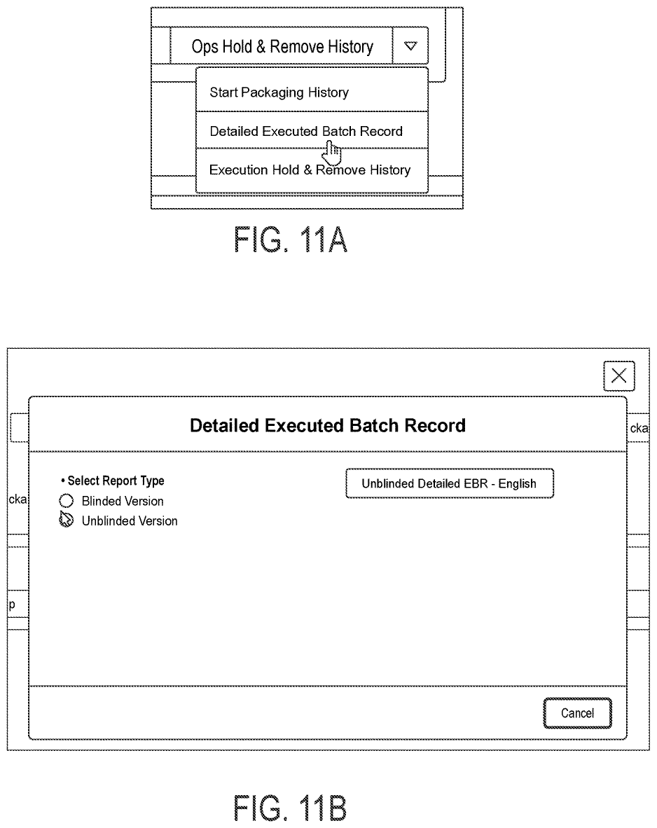

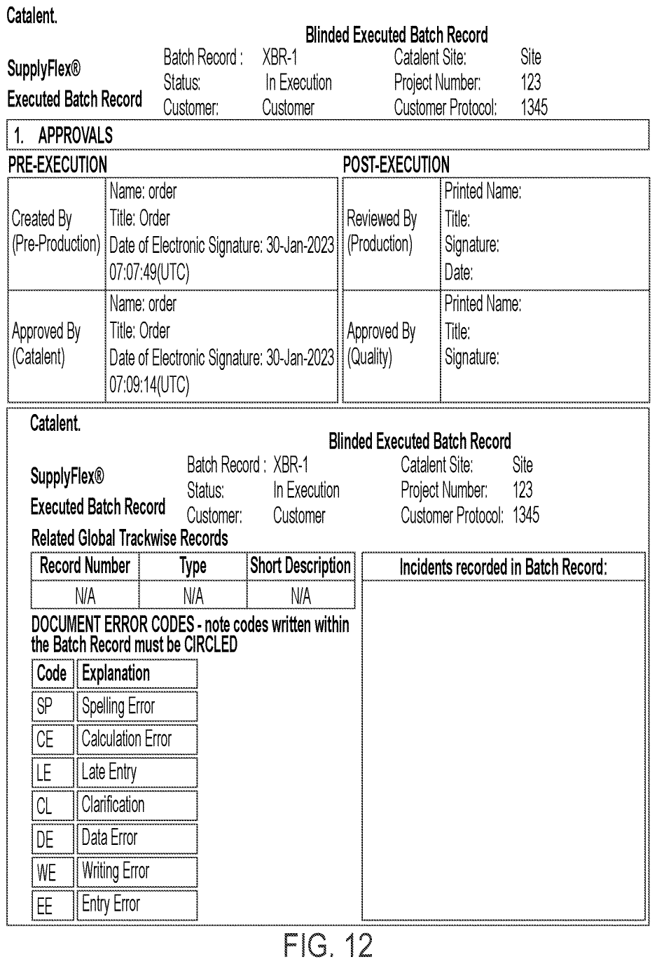

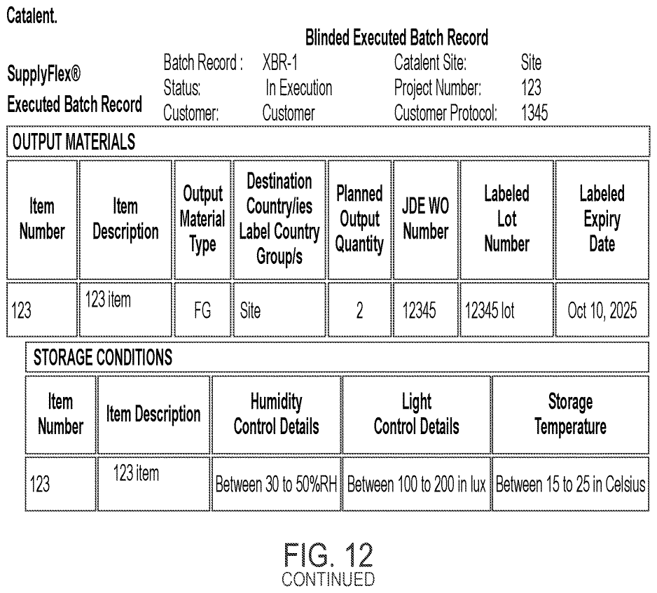

The executed batch record may be subject to both a technical review and a quality review by various users. The executed batch record may be accessed for review by navigating to the Job Actions tab (e.g., Job Actions tab shown in FIG. 9A) and selecting the “Ops Hold & Remove History” button. The system may present a drop-down menu that includes a “Detailed Executed Batch Record” user affordance, as shown in FIG. 11A. Upon receiving a selection of this user affordance, the system can present a pop-up window, as shown in FIG. 11B that allows a user to select whether to view a blinded version or an unblinded version of the detailed executed batch record. Selection of a blinded version of the executed batch record will include one or more redacted fields associated with blinding information. FIG. 12 shows an exemplary excerpt from a blinded executed batch record in accordance with one or more embodiments of this disclosure.

In some examples, the electronic executed batch record can be selected for technical review by users with a first designated access permission (e.g., production reviewer). The technical review may cover the completion and collation of the batch record, as recorded on the batch record contents page, and does not need to be an independent review (i.e., a user who participated in the packaging job may also participate in the technical review). Corrections can be made at any time during the review process. In some embodiments, errors or omissions can be corrected by the user(s) responsible for the initial entry of the erroneous or omitted data. In some embodiments, a second user (e.g., not responsible for the erroneous data) can make the correction. In one or more examples, the user performing the review may read through the contents of the electronic executed batch record, including the approvals of any previous users, for completeness and accuracy.

In some embodiments, the system may display an “Electronic Executed Batch Record Checklist” to support the user performing the review. The steps listed in the “Electronic Executed Batch Record Checklist” may include one or more of the following checking categories: “Batch Record General,” “Approvals,” “Summary,” “Contents,” “Line Clearance,” “Start and End of Production,” “Bill of Materials,” “Material Staging,” “Equipment,” “Briefing,” “Label Sample,” “Packaging Instructions,” “In-Process Control Checklist,” “Reconciliation,” “Batch Record Comments,” “Corrections,” “Blinding Check,” “Batch Record Materials,” “Certificate of Packaging,” “Deviations and Comments,” and “Miscellaneous.” In some embodiments additional categories may be included in the “Electronic Executed Batch Record Checklist.” In some embodiments, the “Electronic Executed Batch Record Checklist” may include one or more user affordances for completing the technical review and one or more user affordances for completing the quality review. Upon completion of the one or more user affordances for completing the technical review of the “Electronic Executed Batch Record Checklist,” the user can enter an e-signature confirming that the electronic executed batch record has been fully reviewed. The user can select the “Submit for Review” button on the Job Actions tab to end the technical review and pass the electronic executed batch record onto the quality review process.

In some examples, the electronic executed batch record can be selected for quality review by users with a second designated access permission (e.g., quality assurance personnel). The quality review may cover the completion, accuracy, and regulatory compliance of the batch record and may be an independent review (i.e., a user or operated who participated in the packaging job may not participate in the quality review). The system may provide the user conducting the quality review with the “Electronic Executed Batch Record Checklist,” as completed during the technical review, which can include one or more user affordances for completing the quality review. If the user performing the quality review identifies issues, the user can request corrections from one or more users with the relevant access permissions (e.g., team lead and/or operator). Once the system receives all identified corrections, the system can receive a user selection of the “Submit for Review History” button on the Job Actions tab. The system can display the e-signature user interface to allow one or more users to e-sign for the completion of the quality review.

The e-signature process may be substantially similar to the e-signature process described above. For example, in response to receiving a selection of the “Approval” button from a user, the system may present the user with one or more text input boxes to enter a user login ID, an individual signature identifier, and/or comments. In one or more embodiments, although the system may be logged into a first user's account, approval and/or e-signature may be attributed to a second user. This is because the system attributes the approval to a user based on the credentials received during the e-sign process instead of automatically attributing a comment to the user that is currently signed into the system. This feature allows packaging teams to work smoothly and cohesively when using embodiments of the present disclosure. For example, multiple users can affix their e-signature to approve the executed batch record without having to sign off of the current account. For example, the system may request approval from more than one user. Having a first user to e-sign, sign-out of the system and the second user sign in to approve the executed batch record may be cumbersome and time consuming. Accordingly, by attributing the e-signature to a user based on credentials entered during the e-signature process as opposed to the account used to complete the packaging run, embodiments of the present disclosure provide an auditable approval trail that improve efficiencies.

Referring back to block 124 of FIG. 1B, the system can release the executed batch record (EBR) upon completion of the technical review and quality review. If applicable, the system may permit the user to perform additional actions at this step, including, but not limited to, changing the expiry date of a product, requesting an additional review of the electronic executed batch record from a Qualified Person (QP), obtaining customer corrections and/or approval, and completing a Certificate of Compliance. The released executed batch record may compile information received by the system from various user interface tabs. In some embodiments, the released executed batch record may correspond to the executed batch record reviewed at block 122. In one or more embodiments, although both a blinded and unblinded version of the executed batch record may be generated, the blinded executed batch record may be released and available to share with the client, while the unblinded executed batch record may not be released nor made available to share with the client.

Referring back to exemplary processes 100A and 100B from FIGS. 1A and 1B, the system may perform additional operations in combination with processes 100A and 100B. These operations may include, but are not limited to, completing an In-Progress Checklist (IPC) for the packaging job, receiving target values for steps in the IPC, recording sample data from the packaging job, entering comments regarding the packaging job, editing the batch record, cancelling the execution of the batch record, and adding attachments to the batch record.

In one or more embodiments, after completing the room clearance and prior to starting the packaging job (i.e., between block 106 and block 116 of FIG. 1A), the system may allow a user to complete actions associated with one or more in-process checklists (IPCs). The in-process checklists may generally relate to checking the input materials in the packaging run. For instance, an IPC may be used to verify blister integrity (e.g., of blister packs), induction seals on bottles, a material count as specified in the batch record, and the like. Generic IPCs may be configured as a part of the master data on the system. The IPC tab may enable a user to select, add, and/or update an in-process checklist and associate the IPC with the batch record in execution. In one or more examples, the checklist can be configured to be executed by one or more users at the beginning, during or toward the end of the packaging process.

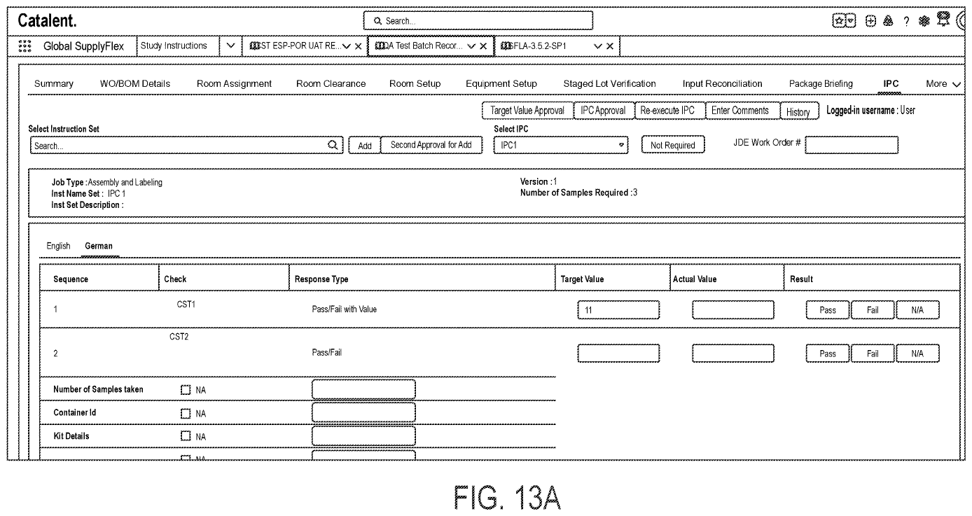

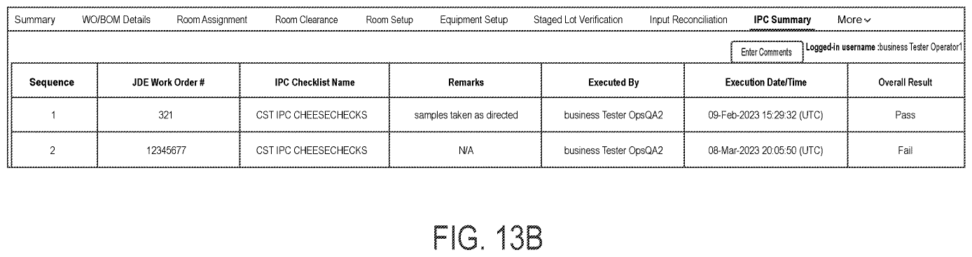

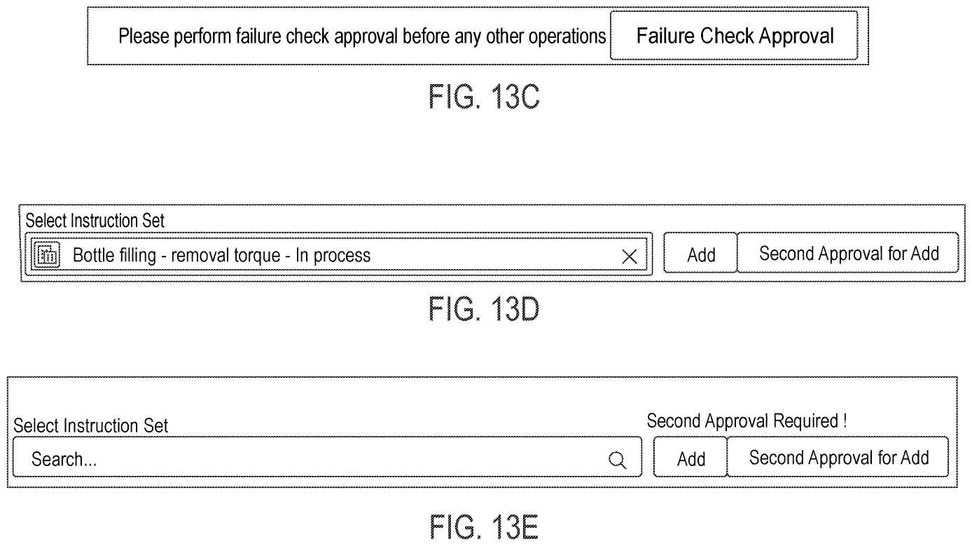

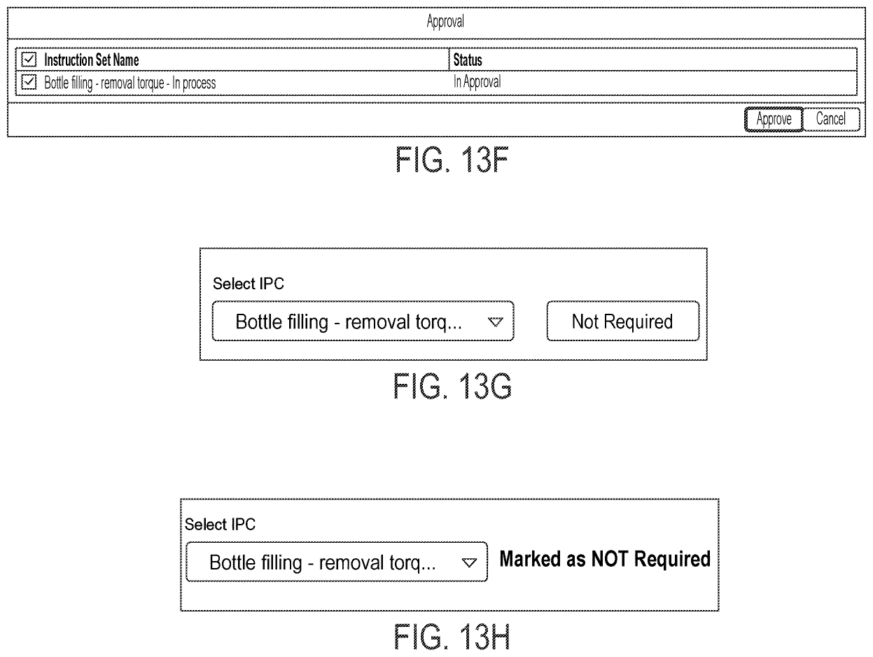

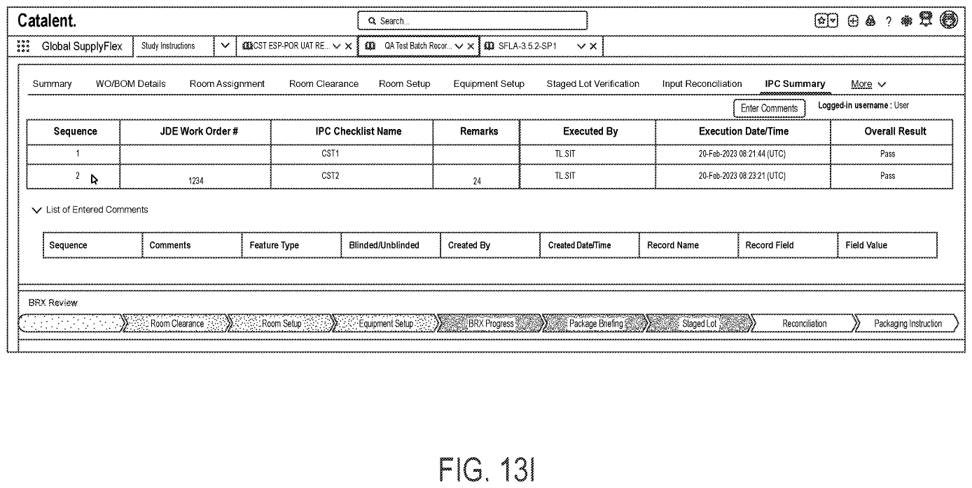

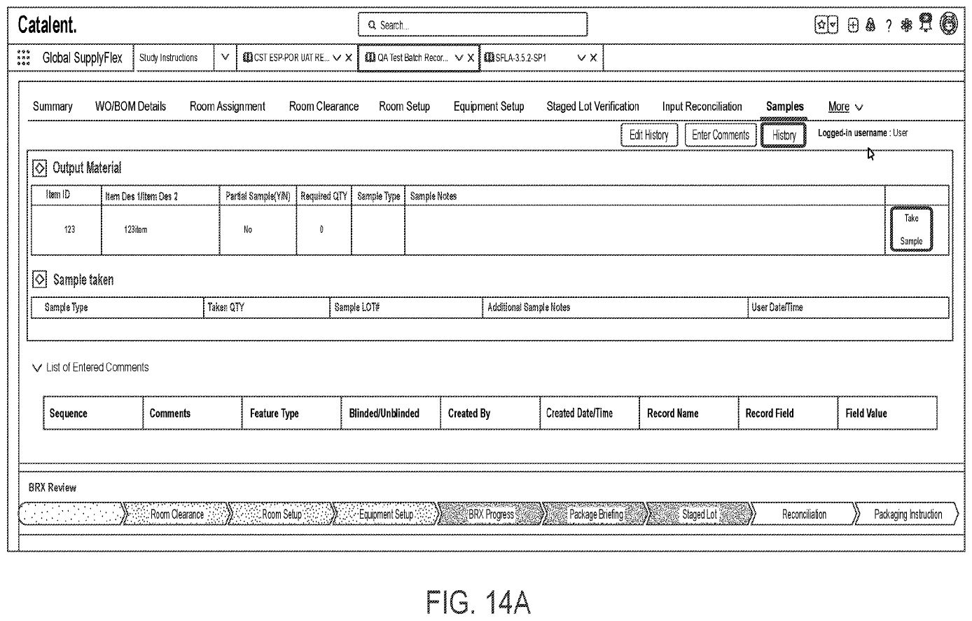

FIG. 13A illustrates an exemplary IPC tab in accordance with embodiments of this disclosure. The system can present an IPC tab and display a corresponding IPC user interface for a user to configure one or more IPCs. In an example, the system may associate instructions to the batch record in execution by receiving a user selection of an existing instruction set associated with the batch record in execution or a user selection of an “Select Instruction Set” option. The “Select Instruction Set” option would allow the user to add one or more instruction sets from the master data.

In one or more examples, an IPC may be associated with one or more target values. For example, a step of the checklist may be associated with a specific value or range of values, e.g., verifying a count of tablets in each bottle as specified in the batch record in execution. As shown in FIG. 13A, the system may present a “Target Value” text input box to receive the IPC target values entered by the user. For example, a user may enter the count of tablets to be included in each bottle as specified in the batch record. The system may receive a user input corresponding to the user selecting a “Target Value Approval” button, to indicate that target values have been entered. The system can then display an e-signature page to receive the user's e-signature for approving the target values input by the user. The system may repeat any of the above steps to receive IPC target values for any additional IPC(s) from the “Select IPC” dropdown menu. In one or more embodiments, the IPC target values may be associated with the checklists generally and updated and saved in the master data. In one or more examples, the IPC target values may be associated with a specific batch record in execution and may not be saved to master data.

The system can receive a selection of a specific IPC by the user and display the IPC to be completed. In some embodiments, a user may additionally enter a work order number in a “Work Order” text input box. As shown in FIG. 13A, the system can display various input fields, including, but not limited to, “Number of samples taken,” “Container ID,” “Kit details,” “Remarks,” “Actual value,” and “Result” (e.g., “Pass,” “Fail,” or “N/A”). The fields shown in FIG. 13A are exemplary, and more or less fields may be displayed and/or associated with an IPC without departing from the scope of this disclosure. The system may receive input values from a user corresponding to one or more of these fields. For example, under the “Actual Value” column, for a particular step of the checklist, a user can enter an actual value observed into a corresponding text box and/or indicate a result, e.g., whether the step has passed, failed, or N/A. For each step of the IPC, the system may request approval. Once the system receives the input in each of the input fields, the system may activate an “IPC Approval” button to receive the user's approval and e-signature. The system may display approved IPCs in the IPC summary tab, as shown in FIG. 13B.

If actual values entered in an IPC are outside of the target values set by the user, the system may prevent the user from completing another IPC. In an example, on the IPC tab, the system may display a “Failure Check Approval” button and a corresponding error message (as shown in FIG. 13C) until a user with a particular access permission (e.g., quality assurance operator) provides input confirming that the failure has been addressed. Upon receiving the user's selection of the “Failure Check Approval” button, the system may receive the user's approval and e-signature. The system may then allow the user (or a different user) to complete another IPC.

In some embodiments, an IPC checklist can be added to the batch record in execution. In some embodiments, an IPC checklist may be added to the batch record in execution before or after the packaging has started (e.g., block 116). For instance, if while on the packaging floor an operator determines that a new checklist should be made, the operator can associate an IPC with the batch record in execution to be completed as a part of packaging run. To add an IPC to the batch record in execution, the system may present a user having a particular access permission (e.g., Team Lead or Ops QA) with a “Select Instruction Set” text input box, which allows the user to search for an IPC to be added, an “Add” button, which allows the user to select the IPC to be added, and a “Second Approval for Add” button, as shown in FIG. 13D. The system can then display an e-signature page to receive the user's e-signature. Upon receiving the user's e-signature, the system may display a “Second Approval Required” notification, as shown in FIG. 13E. If a user selects the “Second Approval for Add” button, the system may display a pop-up window with fields including, but not limited to, IPC name and approval status, as shown in FIG. 13F. Upon receiving the user's selection of an IPC to approve, the system may display an e-signature page to receive the user's e-signature. The e-signature process may be similar to the e-signature processes described above. In some examples, the second approval may be associated with a specific access permission (e.g., quality assurance operator or team lead).

In an example, the system may present the user with an option to mark an IPC as “Not Required” for the batch record in execution. For instance, in the IPC tab, the system may display a “Not Required” button proximate to the “Select IPC” dropdown menu, as shown in FIG. 13G. Upon receiving the user's selection of the IPC to be marked as “Not Required,” the system may display a “Marked as NOT Required” notification associated with the selected IPC, as shown in FIG. 13H. FIG. 13I illustrates an IPC Summary user interface that displays a list of each of the IPCs associated with the batch record in execution.

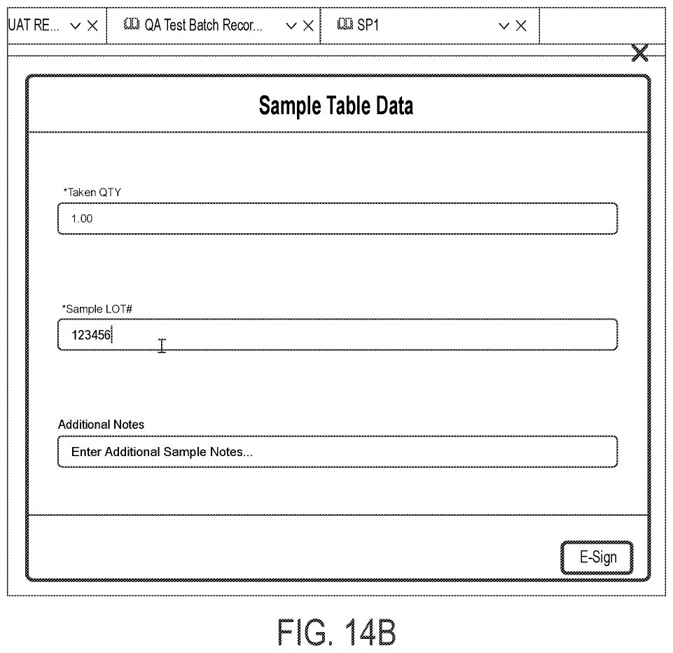

In one or more embodiments, the executable batch record may specify that one or more samples from the packaging run should be obtained. For instance, the executable batch record may specify that four samples should be obtained from the output materials, where two samples should be retained for the client and two samples should be retained at the packaging site as a quality assurance measure or to reference for future packaging runs. The system may receive an input from the user indicating that the user has obtained one or more samples in accordance with the executable batch record. For instance, as shown in FIG. 14A, the system can present a Samples tab including a user interface for a user to enter sample data from the packaging job. If samples are specified as an output material, the system may present a “Take Sample” button under the applicable output material to receive sample data entered by the user. If the user selects the “Take Sample” button, the system may then present the user with various input fields for sample data, including, but not limited to, “Taken QTY,” “Sample Lot #,” and “Additional Notes,” as shown in FIG. 14B. The fields shown in FIG. 14B are exemplary, and more or less information may be associated with the sample data without departing from the scope of this disclosure. As shown in the figure, the user taking the sample may enter an e-signature to enter the sample table data into the system. The system can then record the sample data, and display the initials of the user and the timestamp of the entry on the sample tab. The system may repeat any of the above steps to record sample data for any of the applicable output materials.

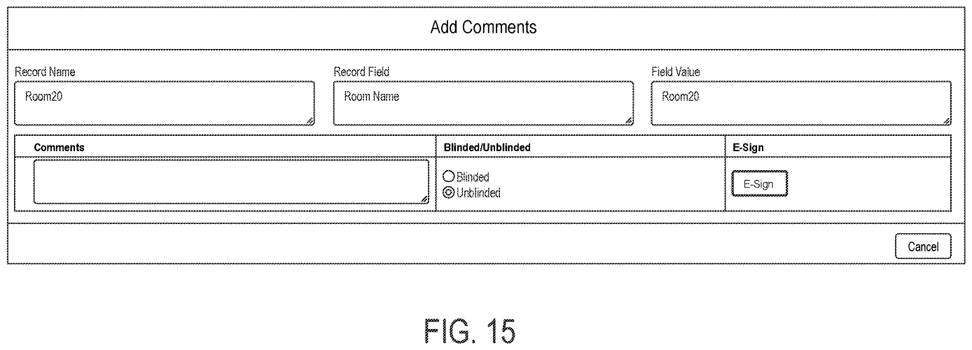

In one or more embodiments, the system may record user comments during the execution of the batch record. The system may record user comments on various tabs of the exemplary user interface, including, but not limited to, Room Assignment, Room Clearance, Room Setup, Equipment Setup, Staged Lot Verification, Package Briefing, Input Reconciliation, Samples, IPC, Packaging Instructions, and Image Capture. These tabs are exemplary, and greater or fewer tabs may be associated with user comments without departing from the scope of this disclosure. On each tab, the system may present an “Enter Comments” button for the user's selection. Upon receiving the selection, the system may display “Field Checkboxes” proximate available fields for which a comment may be entered. The system receives the user's selection of the “Field Checkboxes” and presents the user with an “Add Comments” window. FIG. 15 illustrates an exemplary “Add Comments” window. The “Add Comments” window may include, but is not limited to, the following pre-populated fields associated with the selected “Field Checkboxes”: “Record Name,” “Record Field,” and “Field Value.” Additionally, on the “Add Comments” display, the system may receive comments entered by the user, which can be marked by the user as either “Blinded” or “Unblinded.” The system may display any comments marked as “Unblinded” on the electronic executed batch report. The system may redact any comments marked as “Blinded” on the blinded version of the electronic executed batch report. In response to receiving a user selection of the e-signature button, the system can display an e-signature page to receive the user's credentials. The system may repeat any of the above steps to enter user comments on any of the applicable tabs.

The system may display user comments on each tab under its respective “List of Entered Comments” section. Additionally, the system may display any comments entered across any of the tabs on a Comments Summary tab. In one or more embodiments, the comments may be entered based on the user credentials input in response to selection of the e-Sign button by the user. That is, although a first user may have signed into the system, the comments will not automatically be attributed to the signed-in user account. Instead, the comments will be attributed to the user credentials entered with the e-signature. This feature promotes an accurate auditable history of which users have inputted which comments. For example, because one or more users can input comments throughout the batch record execution without having to sign off of the system associating the comments based on user credentials entered at the time of approval/e-signature ensures that comments are properly attributed to a user and not automatically attributed to a user logged-in to the system.

In one or more embodiments, the system may present the user with options to edit the batch record in execution (BRX) after completing the room assignment (i.e., after block 104 of FIG. 1A). In one or more examples, the batch record edits may be performed while the batch record is on “Execution Hold” (refer to block 116 of FIG. 1A). Upon completion of the edits by a first user having a particular access status (e.g., Pre-Production) and approval of the edits by a second user having a particular access status (e.g., Quality), the system may remove the “Execution Hold” and proceed with the execution of the batch record. The system may present the user with a “BRX Edit” button for editing the batch record on various tabs of the exemplary user interface, including, but not limited to, Summary, Staged Lot Verification, Package Briefing, Samples, and Image Capture. These tabs are exemplary, and greater or fewer tabs may display options to edit the batch record without departing from the scope of this disclosure.

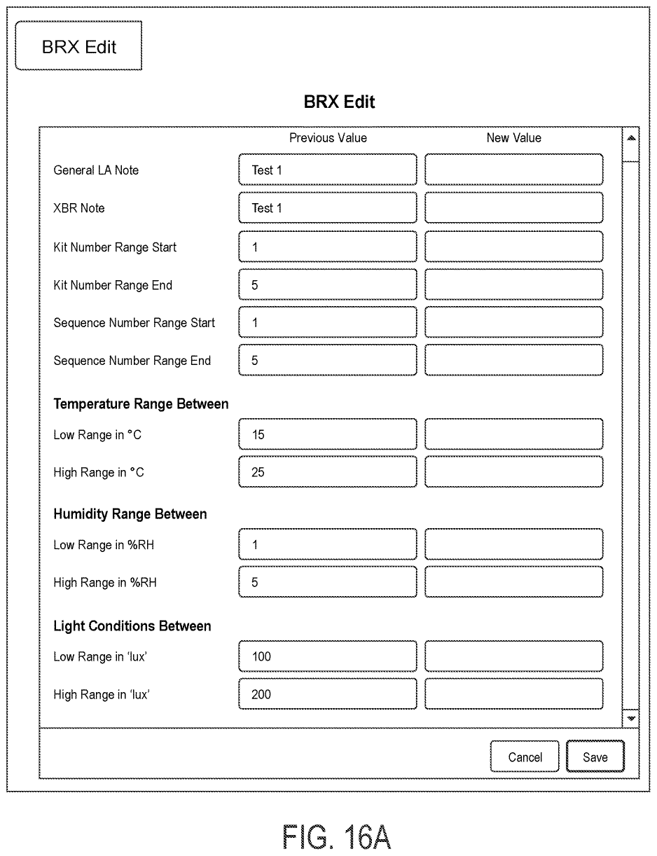

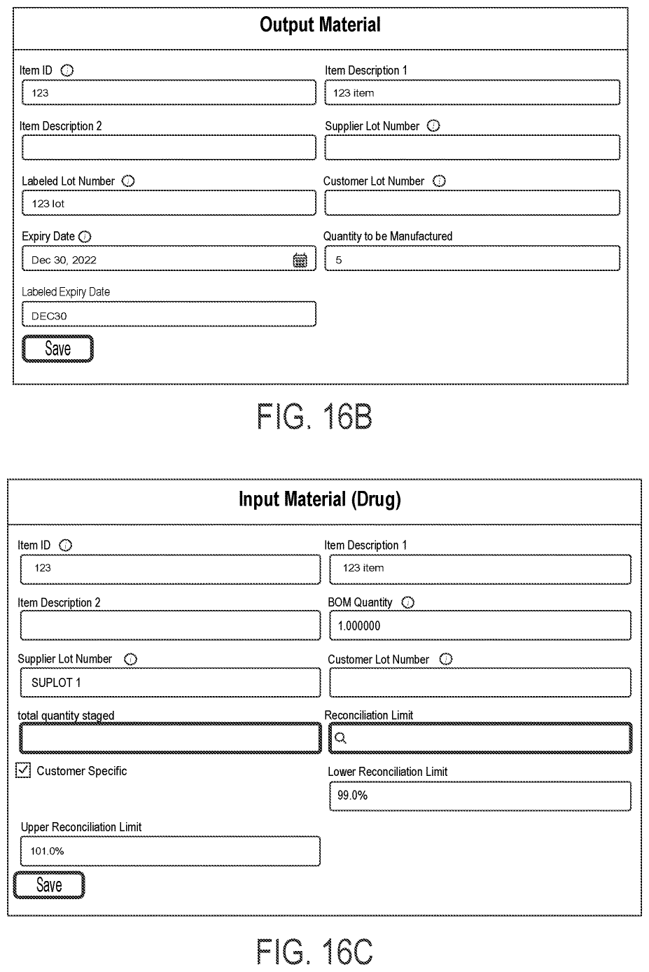

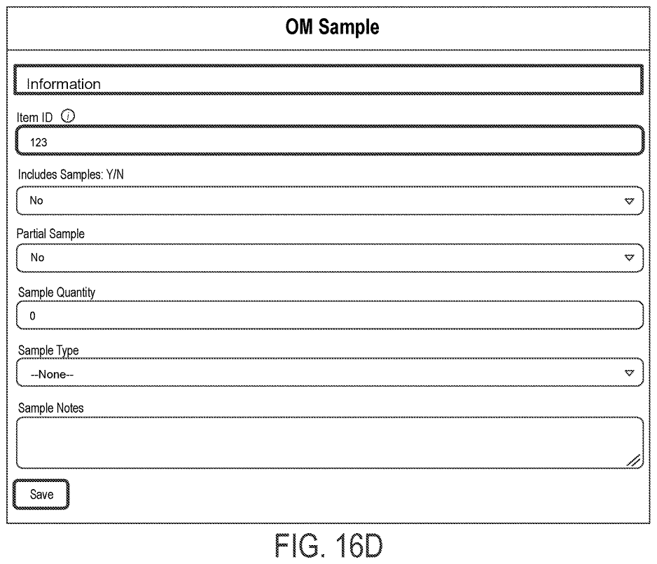

FIG. 16A provides an exemplary “BRX Edit” user interface associated with the Summary tab. In one or more examples, the user interface shown in FIG. 16A may comprise a popup window with one or more editable fields. The “BRX Edit” window may include, but is not limited to, the following fields associated with the Summary tab: general notes, kit number ranges, sequence number ranges, temperature ranges, humidity ranges, and light condition ranges. The system can receive new values for one or more fields. The system can then display an e-signature page to receive the user's e-signature for completion of the edits. In some embodiments, the system may further prompt a second user to provide an e-signature for approval of the edits. This e-signature process may be similar to the e-signature process described above.

FIGS. 16B and 16C illustrate exemplary “BRX Exit” user interfaces for Output Material and Input Material (Drug), respectively, associated with the Staged Lot Verification tab. As shown in the figure, the “BRX Edit” user interface may comprise a popup window with one or more editable fields. These windows may include, but is not limited to, the following fields associated with the input and output materials of Staged Lot Verification tab: item IDs, item descriptions, lot numbers, expiry dates, quantities to be manufactured, BOM quantities, reconciliation limits, and total quantities staged. Upon receiving the first user's edits for the desired fields, the system can then display an e-signature page to receive the first user's e-signature for completion of the edits. In some embodiments, the system may further prompt a second user to provide an e-signature for approval of the edits. This e-signature process may be similar to the e-signature process described above.

The Package Briefing tab may also include a “BRX Edit” user affordance. The system can present a “BRX Edit” popup window with one or more editable fields in response to receiving a user selection of the “BRX Edit” user affordance. The “BRX Edit” window may include, but is not limited to, the following editing features associated with the packaging instructions of the Package Briefing tab: adding and deleting packaging instructions, adding and deleting images associated with a packaging instruction, adding and deleting important notes associated with a packaging instruction, sorting packaging instructions or important notes, and/or adding or updating Label Placements and/or Assembly Drawings. Upon receiving the user's edits, the system can then display an e-signature page to receive the first user's e-signature for completion and approval. In some embodiments, the system may further prompt a second user to provide an e-signature for approval of the edits. This e-signature process may be similar to the e-signature process described above.

FIG. 16D illustrates an exemplary “BRX Edit” pop-up window associated with the Samples tab. Upon receiving a user input selecting the “BRX Edit” user affordance, the system may present a “OM Sample” (e.g., output material sample) popup window with one or more editable fields. The “OM Sample” window may include, but is not limited to, the following fields associated with the Samples tab: item IDs, includes samples (Y/N), partial sample, sample quantity, sample type, and sample notes. Upon receiving the user's edits, the system may receive a user input to save the user edits to the system (e.g., the user may select a “save” user affordance”). The system can then display an e-signature page to receive the user's e-signature for approval of the edits. In some embodiments, the system may further prompt a second user to provide an e-signature for approval of the edits. This e-signature process may be similar to the e-signature process described above.

The Image Capture tab may also include a “BRX Edit” user affordance and present a “BRX Edit” popup window with one or more editable fields. The “BRX Edit” window may include, but is not limited to, the following editing features associated with the image capture instructions on the Image Capture tab: adding/editing/deleting image capture instructions, and sorting image capture instructions into a desired order. Upon receiving the user's edits, the system may receive a user input to save the user edits to the system (e.g., the user may select a “save” user affordance”). The system can then display an e-signature page to receive the user's e-signature for approval of the edits. In some embodiments, the system may further prompt a second user to provide an e-signature for approval of the edits. This e-signature process may be similar to the e-signature process described above.

In one or more embodiments, the system may receive a user input to cancel the execution of the batch record after the reconciliation of input materials has been completed (i.e., after block 118 of FIG. 1A). For instance, the system can present a Job Actions tab including a user interface for a user to perform actions related to a packaging job, e.g., cancelling the execution of the packaging job. In one or more examples, the execution cancellation may be performed prior to starting the packaging run (e.g., step 116). In such examples, the execution cancellation can cancel the batch record in execution without further processing of the batch record. In some examples, the execution cancellation can be approved by two users with two different access permissions (e.g., pre-production operator and team lead). In one or more examples, the pre-production operator can select the “Execution Cancel History” button located on the Job Actions tab (e.g., Job Actions tab shown in FIG. 9A). The system may prompt one or more users to e-sign to approve the execution cancellation. Upon receiving a first user's e-signature, the system may prompt another user with a different access permission (e.g., team lead) to e-sign and complete another approval of the execution cancellation. If a batch record in execution (BRX) is successfully cancelled, it will become read-only, and the system may no longer execute the batch record.

The system may allow a user to attach files to the batch record in execution (BRX) at one or more points during the exemplary processes 100A and 100B from FIGS. 1A and 1B. For instance, on the “Files” section of the Summary tab, the system can present an “Upload File” button. Upon receiving the user's selection of the “Upload File” button, the system may present a popup window allowing the user to drag, drop, and/or select a file. The system may also present a user affordance corresponding to a “Contains Unblinding Information” dropdown menu and receive a selection of “Yes” or “No” indicating whether the file “Contains Unblinding Information.” The system can then attach the file to the batch record in execution. The system may prompt the user to provide an e-signature (e.g., enter user credentials) to approve the file attachment. In one or more examples, the files attached to the batch record in execution may not be displayed in the electronic executed batch record. In one or more embodiments, the files attached to the batch record may be accessible to one or more users when reviewing the executed batch record.

A skilled artisan will understand that these user interfaces are exemplary and more or less information may be included without departing from the scope of this disclosure. Additionally, different display configurations may be used without departing from the scope of this disclosure.

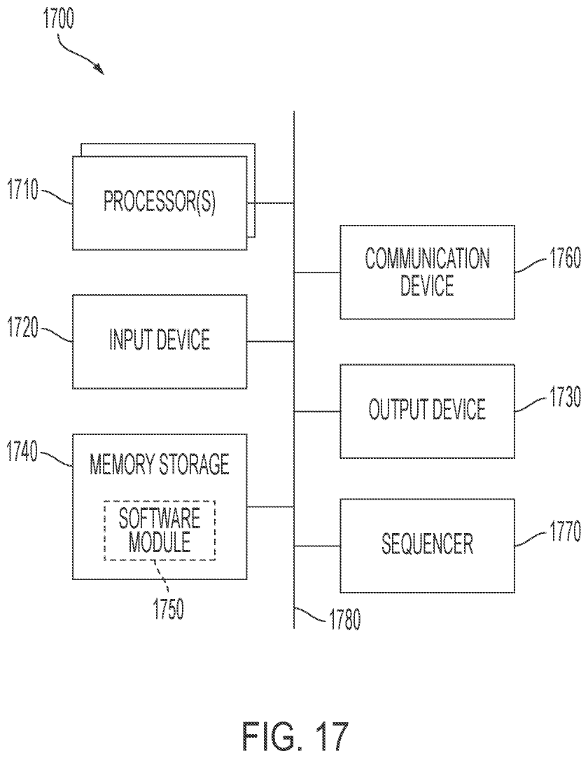

The operations described above with reference to the above-described figures are optionally implemented by components depicted in FIG. 17. It would be clear to a person having ordinary skill in the art how other processes are implemented based on the components depicted in FIG. 17.

FIG. 17 illustrates an example of a computing device in accordance with one embodiment. Device 1700 can be a host computer connected to a network. Device 1700 can be a client computer or a server. As shown in FIG. 17, device 1700 can be any suitable type of microprocessor-based device, such as a personal computer, workstation, server or handheld computing device (portable electronic device) such as a phone or tablet. The device can include, for example, one or more of processor 1710, input device 1720, output device 1730, storage 1740, and communication device 1760. Input device 1720 and output device 1730 can generally correspond to those described above and can be either connectable or integrated with the computer.

Input device 1720 can be any suitable device that provides input, such as a touch screen, keyboard or keypad, mouse, or voice-recognition device. Output device 1730 can be any suitable device that provides output, such as a touch screen, haptics device, or speaker.

Storage 1740 can be any suitable device that provides storage, such as an electrical, magnetic or optical memory including a RAM, cache, hard drive, or removable storage disk. Communication device 1760 can include any suitable device capable of transmitting and receiving signals over a network, such as a network interface chip or device. The components of the computer can be connected in any suitable manner, such as via a physical bus or wirelessly.

Software 1750, which can be stored in storage 1740 and executed by processor 1710, can include, for example, the programming that embodies the functionality of the present disclosure (e.g., as embodied in the devices as described above).

Software 1750 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1740, that can contain or store programming for use by or in connection with an instruction execution system, apparatus, or device.

Software 1750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 1700 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1700 can implement any operating system suitable for operating on the network. Software 1750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

In one or more examples according to the present disclosure, the instructions can further cause the system to receive study information for the clinical trial study, the study information comprising one or more customer approvers, the one or more customer approvers comprising the user associated with the pharmaceutical batch customer. In such examples, the instructions can further cause the system to determine whether the batch record information includes blinding information and, in accordance with a determination that the batch record information comprises blinding information, causing to present a restricted view of the preliminary batch record that omits the blinding information to the user associated with the pharmaceutical batch customer. In such examples, the user associated with the pharmaceutical batch customer can be blinded. In such examples, the blinding information can include a medication list, an identification number, a kit number, a start sequence number, an end sequence number, or a combination thereof. In such examples, a second user associated with the pharmaceutical batch customer can be unblinded. In such examples, the instructions can further cause the system to, in accordance with the determination that the batch record information comprises blinding information, cause to present to the second user associated with the pharmaceutical batch customer, an unrestricted view of the executable batch record that includes the blinding information.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EXEMPLARY EMBODIMENTS

Exemplary implementations of the methods and systems described herein include:
1. A computer implemented method for generating an executed batch record for a clinical trial study, the method comprising:
   receiving an indication that a batch record is assigned to a packaging room located at a packaging site, the packaging room configured to execute the batch record;
   receiving an indication at the packaging site that a first user completed a package briefing associated with a packaging job to execute the batch record;
   in accordance with a determination that the first user completed the package briefing, displaying packaging instructions for producing a quantity of packaged materials for the packaging job;
   determining whether the quantity of packaged materials is within a reconciliation target and updating the batch record to include a determination of whether the quantity of packaged materials is within the reconciliation limit;
   receiving a first approval of the batch record by the first user, the first approval comprising receiving credentials from the first user while logged into an account associated with the first user;
   receiving a second approval of the batch record by a second user, the second approval comprising receiving credentials from the second user while remaining logged-in to the account associated with the first user; and
   generating the executed batch record for the clinical trial study based on the approved batch record.
2. The method of clause 1, wherein the packaging instructions comprise a plurality of steps and the method further comprises: for a step of the plurality of steps, receiving, via an image capture device located in the packaging room, an image comprising a visual indication that the step was completed; and updating the batch record to save the image and associate the image with the step of the plurality of steps.
3. The method of clause 2, comprising for the step of the plurality of steps, receiving an instruction step approval associated with the step by the first user, the instruction step approval comprising receiving the credentials from the first user.
4. The method of any of clauses 1 to 3, comprising:
   obtaining, via an external resource, input material data comprising one or more fields, the materials data associated with an input material staged in the packaging room;
   in accordance with a determination that one or more fields of the input material data matches one or more corresponding fields of the executable batch record, approving the staged input material;
   in accordance with a determination that a field of the input material data does not match a corresponding field of the executable batch record, displaying a user affordance for the first user to accept or reject the input material; and
   in accordance with a determination that the input material is approved or accepted, adding the input material to the batch record in execution.
5. The method of clause 4, wherein the one or more fields of the input material data comprises one or more of a material identification number, a supplier lot number, an external resource lot number, and a customer lot number.
6. The method of clause 4 or clause 5, comprising:
   in accordance with receiving an indication that the first user has accepted the input material, displaying an input material approval interface;
   receiving credentials of the first user via the input material approval interface; and
   accepting or rejecting the input material.
7. The method of any of clauses 1 to 6, further comprising receiving via comment user interface a comment associated with the batch record in execution.
8. The method of clause 7, further comprising receiving a comment approval of the comment by the second user, the comment approval comprising receiving the credentials from the second user while remaining logged-in to the account associated with the first user.
9. The method of any of clause 7 or 8, further comprising receiving an indication that the comment is associated with blinding data.
10. The method of any of clauses 9, wherein generating the executed batch record comprises generating a blinded executed batch record such that the blinding data is redacted.
11. The method of any of clauses 1 to 10, further comprising configuring the packaging room to execute the batch record, wherein configuring the packaging room comprises:
   assigning a packaging room at the packaging site to the packaging job;
   receiving an indication from a user located at the packaging room that the packaging room satisfies one or more items of a room clearance checklist, the room clearance checklist associated the executable batch record and the packaging room;
   receiving an indication from the user that the packaging room satisfies one or more items of a room setup checklist, the room setup checklist associated the executable batch record and the packaging room; and
   receiving an indication from the user that a first equipment located in the packaging room satisfies one or more items of an equipment setup checklist, wherein the equipment setup checklist is associated with the first equipment and the executable batch record.
12. The method of any of clauses 1 to 11, further comprising:
   receiving a user input to put the packaging job on hold;
   in accordance with receiving the user input to put the packaging job on hold, displaying a job hold approval interface;
   receiving credentials of the first user via the job hold approval interface;
   receiving credentials of the second user via the job hold approval interface while remaining logged-in to the account associated with the first user; and
   updating a packaging job status to on hold.
13. The method of any of clauses 1 to 12, further comprising displaying a color-coded progress bar indicative of completion of one or more tasks associated with the packaging job.
14. The method of clause 13, further comprising automatically updating the progress bar when the one or more tasks associated with the packaging job are approved.
15. A method for generating an executed batch record for a clinical trial study, the method comprising:
   at a first computer system comprising one or more processors and one or more memories:
      obtaining an executable batch record for a packaging job, the packaging job configured to performed at a packaging site;
      assigning a packaging room at the packaging site to the packaging job;
      receiving an indication that the packaging room satisfies one or more items of a room clearance checklist, the room clearance checklist associated with the executable batch record and with the packaging room;
      receiving an indication that the packaging room satisfies one or more items of a room setup checklist, the room setup checklist associated with the executable batch record and with the packaging room;
      receiving an indication that a first equipment located in the packaging room satisfies one or more items of an equipment setup checklist, wherein the equipment setup checklist is associated with the first equipment and with the executable batch record;
      receiving an indication that a first user completed a package briefing associated with the packaging job;
      receiving an indication verifying that one or more materials in the packaging room match one or more materials in a bill of materials;
      receiving an indication to start the packaging job;
      in accordance with a determination that the first user completed the package briefing and while logged-in to an account associated with the first user, displaying packaging instructions associated with the packaging run, the packaging instructions comprising a plurality of steps;
      for a step of the plurality of steps, receiving, via an image capture device located in the packaging room, an image comprising a visual indication that the step was completed;
      receiving an input quantity indicative of a quantity of packaged materials;
      verifying that the quantity of packaged materials is within a predetermined reconciliation target;
      receiving an indication that the packaging job is complete;
      receiving a first approval of the batch record by the first user, the first approval comprising receiving credentials from the first user while logged into the account associated with the first user;
      receiving a second approval of the batch record by a second user, the second approval comprising receiving credentials from the second user while remaining logged-in to the account associated with the first user; and
      generating the executed batch record for the clinical trial study based on the approved batch record.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A computer implemented method for generating an executed batch record for a clinical trial study, the method comprising:
receiving an indication that a batch record is assigned to a packaging room located at a packaging site, the packaging room configured to execute the batch record;
receiving an indication at the packaging site that a first user completed a package briefing associated with a packaging job to execute the batch record;
in accordance with a determination that the first user completed the package briefing, displaying packaging instructions for producing a quantity of packaged materials for the packaging job;
determining whether the quantity of packaged materials is within a reconciliation target and updating the batch record to include a determination of whether the quantity of packaged materials is within the reconciliation limit;
receiving a first approval of the batch record by the first user, the first approval comprising receiving credentials from the first user while logged into an account associated with the first user;
receiving a second approval of the batch record by a second user, the second approval comprising receiving credentials from the second user while remaining logged-in to the account associated with the first user; and
generating the executed batch record for the clinical trial study based on the approved batch record.

2. The method of claim 1, wherein the packaging instructions comprise a plurality of steps and the method further comprises: for a step of the plurality of steps, receiving, via an image capture device located in the packaging room, an image comprising a visual indication that the step was completed; and updating the batch record to save the image and associate the image with the step of the plurality of steps.

3. The method of claim 2, comprising for the step of the plurality of steps, receiving an instruction step approval associated with the step by the first user, the instruction step approval comprising receiving the credentials from the first user.

4. The method of claim 1, comprising:
obtaining, via an external resource, input material data comprising one or more fields, the materials data associated with an input material staged in the packaging room;
in accordance with a determination that one or more fields of the input material data matches one or more corresponding fields of the executable batch record, approving the staged input material;
in accordance with a determination that a field of the input material data does not match a corresponding field of the executable batch record, displaying a user affordance for the first user to accept or reject the input material; and
in accordance with a determination that the input material is approved or accepted, adding the input material to the batch record in execution.

5. The method of claim 4, wherein the one or more fields of the input material data comprises one or more of a material identification number, a supplier lot number, an external resource lot number, and a customer lot number.

6. The method of claim 4, comprising:
in accordance with receiving an indication that the first user has accepted the input material, displaying an input material approval interface;
receiving credentials of the first user via the input material approval interface; and
accepting or rejecting the input material.

7. The method of claim 1, further comprising receiving via comment user interface a comment associated with the batch record in execution.

8. The method of claim 7, further comprising receiving a comment approval of the comment by the second user, the comment approval comprising receiving the credentials from the second user while remaining logged-in to the account associated with the first user.

9. The method of claim 7, further comprising receiving an indication that the comment is associated with blinding data.

10. The method of claim 9, wherein generating the executed batch record comprises generating a blinded executed batch record such that the blinding data is redacted.

11. The method of claim 1, further comprising configuring the packaging room to execute the batch record, wherein configuring the packaging room comprises:
assigning a packaging room at the packaging site to the packaging job;
receiving an indication from a user located at the packaging room that the packaging room satisfies one or more items of a room clearance checklist, the room clearance checklist associated the executable batch record and the packaging room;
receiving an indication from the user that the packaging room satisfies one or more items of a room setup checklist, the room setup checklist associated the executable batch record and the packaging room; and
receiving an indication from the user that a first equipment located in the packaging room satisfies one or more items of an equipment setup checklist, wherein the equipment setup checklist is associated with the first equipment and the executable batch record.

12. The method of claim 1, further comprising:
receiving a user input to put the packaging job on hold;
in accordance with receiving the user input to put the packaging job on hold, displaying a job hold approval interface;
receiving credentials of the first user via the job hold approval interface;
receiving credentials of the second user via the job hold approval interface while remaining logged-in to the account associated with the first user; and
updating a packaging job status to on hold.

13. The method of claim 1, further comprising displaying a color-coded progress bar indicative of completion of one or more tasks associated with the packaging job.

14. The method of claim 13, further comprising automatically updating the progress bar when the one or more tasks associated with the packaging job are approved.

15. A method for generating an executed batch record for a clinical trial study, the method comprising:
at a first computer system comprising one or more processors and one or more memories:
obtaining an executable batch record for a packaging job, the packaging job configured to performed at a packaging site;
assigning a packaging room at the packaging site to the packaging job;
receiving an indication that the packaging room satisfies one or more items of a room clearance checklist, the room clearance checklist associated with the executable batch record and with the packaging room;
receiving an indication that the packaging room satisfies one or more items of a room setup checklist, the room setup checklist associated with the executable batch record and with the packaging room;
receiving an indication that a first equipment located in the packaging room satisfies one or more items of an equipment setup checklist, the equipment setup checklist associated with the first equipment and with the executable batch record;
receiving an indication that a first user completed a package briefing associated with the packaging job;
receiving an indication verifying that one or more materials in the packaging room match one or more materials in a bill of materials;
receiving an indication to start the packaging job;
in accordance with a determination that the first user completed the package briefing and while logged-in to an account associated with the first user, displaying packaging instructions associated with the packaging run, the packaging instructions comprising a plurality of steps;
for a step of the plurality of steps, receiving, via an image capture device located in the packaging room, an image comprising a visual indication that the step was completed;
receiving an input quantity indicative of a quantity of packaged materials;
verifying that the quantity of packaged materials is within a predetermined reconciliation target;
receiving an indication that the packaging job is complete;
receiving a first approval of the batch record by the first user, the first approval comprising receiving credentials from the first user while logged into the account associated with the first user;
receiving a second approval of the batch record by a second user, the second approval comprising receiving credentials from the second user while remaining logged-in to the account associated with the first user; and
generating the executed batch record for the clinical trial study based on the approved batch record.
